SEQUENCE LISTING (15 sequences)

SEQ ID NO: 1 - protein, 22 aa, Artificial sequence
  ER signal peptide
MKTNLFLFLIFSLLLSLSSAEF

SEQ ID NO: 2 - protein, 7 aa, Artificial sequence
  Vacuolar targeting signal from Tobacco chitinase A
DLLVDTM

SEQ ID NO: 3 - DNA, 1491 bp, Homo sapiens
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccag

SEQ ID NO: 4 - protein, 497 aa, Artificial sequence [sequence omitted]

SEQ ID NO: 5 - DNA, 338 bp, Cauliflower mosaic virus
ttttcacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 6 - DNA, 66 bp, Artificial sequence
  Nucleic acid sequence encoding the ER signal peptide
atgaagactaatctttttctctttctcatcttttcacttctcctatcattatcctcggccgaattc

SEQ ID NO: 7 - DNA, 21 bp, Artificial sequence
  Nucleic acid sequence encoding the vacuolar targeting sequence
gatcttttagtcgatactatg

SEQ ID NO: 8 - DNA, 167 bp, Artificial sequence
  Nucleic acid sequence of the Agrobacterium tumefaciens terminator
taatttcatgatctgttttgttgtattcccttgcaatgcagggcctagggctatgaataaagttaatgtgtgaatgtgtgaatgtgtgattgtgacctgaagggatcacgactataatcgtttataataaacaaagactttgtcccaaaaaccccccccccngcaga

SEQ ID NO: 9 - DNA, 2186 bp, Artificial sequence
  nucleic acid sequence encoding high mannose human glucocerebrosidase (GCD)
ttttcacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgagggatccaaggagatataacaatgaagactaatctttttctctttctcatcttttcacttctcctatcattatcctcggccgaattcgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccaagatcttttagtcgatactatgtaatttcatgatctgttttgttgtattcccttgcaatgcagggcctagggctatgaataaagttaatgtgtgaatgtgtgaatgtgtgattgtgacctgaagggatcacgactataatcgtttataataaacaaagactttgtcccaaaaaccccccccccngcaga

SEQ ID NO: 10 - protein, 526 aa, Artificial sequence
  High mannose human glucocerebrosidase (GCD)
MKTNLFLFLIFSLLLSLSSAEFARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQDLLVDTM

SEQ ID NO: 11 - protein, 506 aa, Artificial sequence
  Processed plant produced human recombinant GCD protein
EFARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQDLLVDTM

SEQ ID NO: 12 - protein, 20 aa, Artificial sequence
  ER signal peptide
MKTNLFLFLIFSLLLSLSSA

SEQ ID NO: 13 - protein, 497 aa, Homo sapiens
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 14 - protein, 536 aa, Artificial sequence
  Human GCD fused to an artificial leader sequence
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 15 - DNA, 1491 bp, Homo sapiens
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccag

(19) United States
(12) Patent Application Publication
(10) Pub. No.: US 2017/0101633 A1
(43) Pub. Date: Apr. 13, 2017

(54) METHOD OF MAINTAINING DISEASE STABILITY IN A SUBJECT HAVING GAUCHER'S DISEASE

(71) Applicant: PROTALIX LTD., Carmiel (IL)

(72) Inventors: Yoseph SHAALTIEL, Timrat (IL); Einat ALMON, Timrat (IL)

(21) Appl. No.: 15/117,826

(22) PCT Filed: Feb. 9, 2015
(86) PCT No.: PCT/IL2015/050145

§ 371 Date: Aug. 10, 2016

Related U.S. Application Data
(60) Provisional application No. 61/937,901, filed on Feb. 10, 2014.

(51) Int. Cl. C12N 9/42

(52) U.S. Cl. CPC C12N 9/2434; C12Y 302/01045; A61K 38/00

(57) ABSTRACT

A method of maintaining disease stability in a subject having Gaucher's disease following switch from enzyme replacement therapy (ERT) is provided. The method comprising orally administering to the subject a therapeutically effective amount of recombinant glucocerebrosidase (GCD) comprised in plant cells, thereby maintaining disease stability following switch.

### FIELD AND BACKGROUND OF THE INVENTION

The present invention, in some embodiments thereof, relates to a method of maintaining disease stability in a subject having Gaucher's disease.

Gaucher disease is the most prevalent lysosomal storage disorder. It is caused by a recessive genetic disorder (chromosome 1 q21-q31) resulting in deficiency of glucocerebrosidase, also known as glucosylceramidase, which is a membrane-bound lysosomal enzyme that catalyzes the hydrolysis of the glycosphingolipid glucocerebroside (glucosylceramide, GlcCer) to glucose and ceramide. Gaucher disease is caused by point mutations in the hGCD (human glucocerebrosidase) gene (GBA), which result in accumulation of GlcCer in the lysosomes of macrophages. The characteristic storage cells, called Gaucher cells, are found in liver, spleen and bone marrow. The associated clinical symptoms include severe hepatosplenomegaly, anemia, thrombocytopenia and skeletal deterioration.

Replacement of the missing lysosomal enzyme with exogenous biologically active enzyme has been suggested in the 1960s as a viable approach to treatment of lysosomal storage diseases. Since that time, various studies have suggested that enzyme replacement therapy may be beneficial for treating various lysosomal storage diseases. The best success has been shown with individuals with type I Gaucher disease, first treated with purified placenta GCD(Ceredase™) or, more recently, with recombinantly produced GCD (available from Genzyme Inc., Shire plc., and Protalix Biotherapeutics). All these drugs are administered intravenously and are effective in improving Gaucher's patient's quality of life.

One of the current goals in treatment of Gaucher's patients is to reduce the immense costs associated with ERT as well as to improve patient's life quality by negating frequent visits at the health center and obviating infusion-related complications.

Thus devising therapeutic modalities for maintenance therapy in patients with Gaucher disease is pivotal.

### RELATED BACKGROUND ART

WO2004/096978 WO2007/010533 and WO2013/121405 teach a naturally encapsulated plant cell expressed form of GCD for the treatment of Gaucher disease via oral administration.

Elstein et al. Blood 2007 vol. 110 no. 7 2296-2301.

### SUMMARY OF THE INVENTION

According to an aspect of some embodiments of the present invention there is provided a method of maintaining disease stability in a subject having Gaucher's disease following switch from enzyme replacement therapy (ERT), the method comprising orally administering to the subject a therapeutically effective amount of recombinant glucocerebrosidase (GCD) comprised in plant cells, thereby maintaining disease stability following switch.

According to an aspect of some embodiments of the present invention there is provided a use of a therapeutically effective amount of recombinant glucocerebrosidase (GCD) comprised in plant cells in the manufacture of a medicament identified for maintaining disease stability in a subject having Gaucher's disease after a switch from enzyme replacement therapy (ERT).

According to some embodiments of the invention, the method further comprises periodically testing at least one of liver volume, spleen volume and complete blood count.

According to some embodiments of the invention, the subject has been treated with ERT for at least 2 years, with a stable dose regimen for at least last 6 months before the switch.

According to some embodiments of the invention, the subject exhibits clinically and biologically stable disease for the last 12 months prior to the switch as manifested by stable organomegaly, no progressive symptomatic documented bone disease, no major surgery, hemoglobin level >11 g/dl, mean platelet count >120,000/μL±40%, mean platelet count ≦120,000/μL±20%, chitotriosidase activity within 20% of the mean, no blood transfusion or major bleeding, no acute avascular necrosis event.

According to some embodiments of the invention, the stable organomegaly is manifested by liver volume within 10% of the mean and spleen volume within 10% of the mean.

According to some embodiments of the invention, the Gaucher's disease is type I or type III Gaucher's disease.

According to some embodiments of the invention, the therapeutically effective amount of GCD corresponds to 1-1920 units/Kg/14 days.

According to some embodiments of the invention, the therapeutically effective amount of GCD corresponds to 50-150 units/Kg/day, thereby treating Gaucher's disease. According to some embodiments of the invention, the administering is performed preprandially or over a light meal such that the stomach pH is above 2, thereby treating Gaucher's disease.

According to some embodiments of the invention, the administering is effected daily.

According to some embodiments of the invention, the administering is performed preprandially.

According to some embodiments of the invention, the administering is effected following light meal such that the stomach pH of the subject is above 2.

According to some embodiments of the invention, the cells comprise carrot cells.

According to some embodiments of the invention, the administering is performed once a day.

According to some embodiments of the invention, the administering is performed twice a day.

According to some embodiments of the invention, the administering is performed three times a day.

According to some embodiments of the invention, the administering is performed four times a day.

According to some embodiments of the invention, the plant cells comprise lyophilized plant cells.

According to some embodiments of the invention, the glucocerebrosidase is human glucocerebrosidase.

According to some embodiments of the invention, the glucocerebrosidase is as set forth in SEQ ID NO: 4 or 13.

According to some embodiments of the invention, the human glucocerebrosidase protein is linked at its N terminus to an endoplasmic reticulum signal peptide.

According to some embodiments of the invention, the endoplasmic reticulum signal peptide is as set forth in SEQ ID NO: 1 or 12.

According to some embodiments of the invention, the human glucocerebrosidase protein is linked at its C terminus to vacuolar signal peptide. According to some embodiments of the invention, the vacuolar signal peptide is as set forth in SEQ ID NO: 2.

According to some embodiments of the invention, the glucocerebrosidase has an increased affinity for, and uptake into macrophages, in comparison with the corresponding affinity and uptake of a recombinant human glucocerebrosidase protein produced in mammalian cells, and having glucocerebrosidase catalytic activity.

According to some embodiments of the invention, the main glycan structure of the glucocerebrosidase of the plant cells comprises at least one xylose residue and at least one exposed mannose residue, as measured by linkage analysis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration of a theoretical assumption of the efficacy of enzyme replacement therapy as achieved using a bolus intravenous injection (full line) or by oral daily administration (dashed line). The standard IV treatment given is based on an accumulation of the GCD substrate (glucosylceramide) during two weeks and then a bolus dose that brings it down to the basic level. Without being bound to theory, it is believed that administering the enzyme orally will enable daily treatment that will keep the substrate on its basic level.

FIG. 2 stability over time of plant recombinant (pr)GCD from lyophilized carrot cells expressing same in −20° C., 4° C. and 25° C.

FIGS. 3A-B show that prGCD is able to cross the intestinal barrier FIG. 3A—is an illustration demonstrating the transcytosis assay with prGCD. The assay mimics intestinal translocation. FIG. 3B—prGCD is added to the apical chamber in simulated intestinal medium at 6.8 units /ml. Transcytosis is measured at the basolateral medium after the indicated times at 37° C. Clearly prGCD crosses the simulated epithelial barrier with an apparent permeability coefficient of 1.39·10⁻⁷ cm/sec.

FIGS. 4A-D are images and graphs showing the timeline of carrot cells and prGCD activity passing through the GIT (Numbers indicate time in hours post feeding).

FIGS. 4A-B show images of stomach filled with fed carrot cells (FIG. 4A) and reduction of stomach content weight (gr) in same along time (FIG. 4B). FIGS. 4C-D are graphs showing prGCD activity in the content of the rat GIT (stomach and colon, in mUnits/gr tissue) (FIG. 4C) and in organs (plasma and liver, in mUnits/gr tissue, FIG. 4D).

FIG. 5 shows prGCD survival in purified form and in cells, in the extreme environment of simulated gastric fluid. Note that prGCD activity in cells resists a wider pH range.

FIG. 6 is a bar graph showing prGCD activity in medium and in cells containing prGCD, following treatment of the cells with simulated intestinal media mimicking fasted and fed conditions.

FIGS. 7A-C are bar graphs showing that active prGCD is found in target organs (spleen and liver) after feeding in rats in comparison to injected prGCD. FIG. 7A shows prGCD activity in liver and spleen (in fold increase over average baseline) after feeding of carrot cells with or without (control—) prGCD. FIGS. 7B-C show the percentage of prGCD activity, from the total fed GCD that is measured in the target organ (FIG. 7B) as compared to the percentage of prGCD activity, from the total injected GCD that is measured in the target organ (FIG. 7C).

FIGS. 8A-B are graphs showing GCD activity in leukocytes of whole blood or liver of rats fed with carrot cells expressing human recombinant GCD. Rats (n=21) were fed with carrot cells twice with a six hours interval. Whole blood samples were taken at the indicated time points and the red blood cells were lysed and removed. The leukocytes were extracted and tested for their GCD activity (FIG. 8A). The rats were then sacrificed and their livers were extracted and tested for GCD activity, compared with naïve rats (n=3, FIG. 8B).

FIGS. 9A-B are graphs showing GCD activity in the plasma (FIG. 9A) or livers (FIG. 9B) of pigs fed with carrot cells expressing human recombinant GCD. Pigs (n=3) were fed with carrot cells once. Plasma samples were taken at the indicated time points and tested for their GCD activity (FIG. 9A). The pigs were then sacrificed and their livers were extracted and tested for GCD activity, compared with naïve pigs (n=5, FIG. 9B).

FIGS. 10A-B are graphs showing Cmax of GCD activity in leukocytes following orally administration of carrot cells expressing plant recombinant GCD (prGCD).

FIGS. 11A-F are graphs showing pharmacokinetic profiles of representative patients following 1 day of administration, 3 days of administration and mean baseline levels.

FIG. 12 is a Table showing complete blood count of a naïve Gaucher patient (not receiving ERT) prior to and following treatment with 250 U of orally administered prGCD comprised in carrot cells.

FIG. 13 is a Table showing a prominent clinical effect of oral prGCD on thrombocytopenia.

### DESCRIPTION OF SPECIFIC EMBODIMENTS OF THE INVENTION

The present invention, in some embodiments thereof, relates to a method of maintaining disease stability in a subject having Gaucher's disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Gaucher's disease is an inherited, genetic lysosomal storage disorder caused by mutations or a deficiency of the enzyme GCD. The disease causes harmful accumulations of lipids in the spleen, liver, lungs and brain, and affects patients' bones, bone marrow and platelet count.

Replacement of the missing lysosomal enzyme, GCD, with exogenous biologically active enzyme has been suggested in the 1960s as a viable approach to treatment of lysosomal storage diseases. This approach is known as “enzyme replacement therapy” (ERT), whereby the enzyme is typically administered intravenously.

One of the current goals in treatment of Gaucher's patients is to reduce the immense costs associated with ERT as well as to improve patient's life quality by negating frequent visits at the health center and obviating infusion-related complications.

Thus devising therapeutic modalities for maintenance therapy in patients with Gaucher disease is pivotal.

Whilst conceiving the present invention, the present inventors have realized that oral administration of recombinant GCD comprised in plant cells (i.e., expressed but not secreted) can be efficaciously and safely administered to Gaucher's patients after a switch from ERT in order to maintain disease stability.

Thus, according to an aspect of the invention there is provided a method of maintaining disease stability in a subject having Gaucher's disease following switch from enzyme replacement therapy (ERT), the method comprising orally administering to the subject a therapeutically effective amount of recombinant glucocerebrosidase (GCD) comprised in plant cells, thereby maintaining disease stability following switch.

Accordingly there is provided a use of a therapeutically effective amount of recombinant glucocerebrosidase (GCD) comprised in plant cells in the manufacture of a medicament identified for maintaining disease stability in a subject having Gaucher's disease after a switch from enzyme replacement therapy (ERT).

Thus the subject is afflicted with Gaucher's disease (e.g., type I or type III, as further described hereinbelow) diagnosed by glucocerebrosidase (GCD) activity assay and optionally molecular analysis of the GCD gene.

According to a specific embodiment the subject has been treated with ERT for at least 0.5 years prior to said switch. According to a specific embodiment, the patient has been treated with ERT for 0.5-40 years, 0.5-30 years, 0.5-20 years, 0.5-10 years, 0.5-5 years, 0.5-4 years, 0.5-3 years or 0.5-2 years, or 1-2 or 0.5-1 e.g., 2-3 years. An exemplary regimen is a stable dose regimen for at least the last 6 months before the switch, typically including a bi-weekly administration mode.

As used herein “maintaining disease stability” refers to maintaining clinical and biological parameters achieved after at least two years on ERT. Examples of such parameters are provided infra.

As used herein the phrase “following switch from enzyme replacement therapy” refers to termination of ERT and switching to another treatment modality, in this case, administration of GCD comprised in plant cells, as further described hereinbelow.

Determining patient's stability is typically effected for a number of weeks prior to the switch, e.g., 12 weeks, also termed as the stability period. According to an embodiment of the invention the subject is under ERT treatment at the stability period.

Examples of qualification criteria as well as clinical and biological parameters for determining a stable disease under ERT include, but are not limited to:

Clinical and biological parameters for determining disease stability are typically based on a plurality of measurements at various time points e.g., with at least two time points assessments:

Enzymatic activity GCD enzymatic activity (in peripheral leukocytes or dermal fibroblasts) is determined using methods which are well known to the skilled artisan.

Stable organomegaly can be determined by palpation or imaging such as using ultrasound, magnetic resonance imaging (MRI) or computed tomography (CT).

Exemplary parameters for organomegaly include:

Liver volume within 10% of the mean;

Spleen volume within 10% of the mean.

Free of progressive symptomatic documented bone disease;

Hemoglobin levels within 15% of the mean, e.g., >11 g/dL;

Mean platelet count >120,000/μL±40%,

Mean platelet count ≦120,000/μL±20%, chitotriosidase activity within 20% of the mean. Other relevant biomarkers include but are not limited to angiotensin converting enzyme (ACE), tartrate resistant acid phosphatase (TRAP) and ferritin;

No blood transfusion or major bleeding;

No acute avascular necrosis event.

As used herein “Gaucher's disease” or “Gaucher disease” refers a genetic disease in which a fatty substance (lipid) accumulates in cells and certain organs. Gaucher disease is the most common of the lysosomal storage diseases. It is caused by a hereditary deficiency of the enzyme glucocerebrosidase (also known as acid β-glucosidase). The enzyme acts on a fatty substance glucocerebroside (also known as glucosylceramide). When the enzyme is defective, glucocerebroside accumulates, particularly in white blood cells (mononuclear leukocytes). Glucocerebroside can collect in the spleen, liver, kidneys, lungs, brain and bone marrow.

Gaucher's disease has three common clinical subtypes.

Type I (or non-neuropathic type, GD1) is the most common form of the disease, occurring in approximately 1 in 50,000 live births. It occurs most often among persons of Ashkenazi Jewish heritage. Symptoms may begin early in life or in adulthood and include enlarged liver and grossly enlarged spleen (together hepatosplenomegaly); the spleen can rupture and cause additional complications. Spleen enlargement and bone marrow replacement cause anemia, thrombocytopenia and leukopenia. Skeletal weakness and bone disease may be extensive. The brain is not affected pathologically, but there may be lung and, rarely, kidney impairment. Patients in this group usually bruise easily (due to low levels of platelets) and experience fatigue due to low numbers of red blood cells. Depending on disease onset and severity, type 1 patients may live well into adulthood. Some patients have a mild form of the disease or may not show any symptoms.

Type II (or acute infantile neuropathic Gaucher disease, GD2) typically begins within 6 months of birth and has an incidence rate of approximately 1 in 100,000 live births. Symptoms include an enlarged liver and spleen, extensive and progressive brain damage, eye movement disorders, spasticity, seizures, limb rigidity, and a poor ability to suck and swallow. Affected children usually die by age of 2.

Type III (the chronic neuropathic form, GD3) can begin at any time in childhood or even in adulthood, and occurs in approximately 1 in 100,000 live births. It is characterized by slowly progressive but milder neurologic symptoms compared to the acute or type 2 version. Major symptoms include an enlarged spleen and/or liver, seizures, poor coordination, skeletal irregularities, eye movement disorders, blood disorders including anemia and respiratory problems. Patients often live into their early teen years and adulthood.

According to a specific embodiment, the Gaucher's disease is type I or type III.

As used herein “enzyme replacement therapy (ERT)” refers to the exogenous administration of glucocerebrosidase (GCD). The enzyme is not comprised in plant cells. A number of health regulatory agency-approved versions of GCD are available on the market. Examples include, but are not limited to, Elelyso (taliglucerase), Cerezyme (imiglucerase), Vpriv (velaglucerase) and Ceredase (alglucerase).

Modes of administration of oral GCD encapsulated in plant cells are provided infra.

According to an embodiment of the invention, the method comprising orally administering to the subject a therapeutically effective amount of recombinant glucocerebrosidase (GCD) comprised in plant cells, wherein the therapeutically effective amount of GCD corresponds to 1-1920 units/Kg/14 days e.g., 40-1920 units/Kg/14 days, 50-150 units/Kg/14 days, e.g., 50 units/Kg/14 days, 100 units/Kg/14 days or 150 units/Kg/14 days.

As used herein the term “corresponds” refers to the full dose administered over a period of two weeks. The administration can be low frequency bolus administration (e.g., biweekly). Alternatively, administration is effected at low doses and higher frequency. Thus administration the above-mentioned enzyme dose can be daily, every two days, every three days, occur twice a week. It will be appreciated that the inclusion of GCD in plant cells and its oral administration results in a slow-release-like effect, whereby the enzyme is slowly released to the circulation (digestion-dependent), thus maintaining essentially constant levels of the enzyme in the blood. High frequency of administration (relative to the i.v. route) ensures maintenance of effective levels of enzymes in the circulation. Thus, the administration of the enzyme in the plant cells, high frequency administration or the combination of same may allow reducing the overall dose of the enzyme administered (again, in comparison to the i.v. administered doses).

According to an additional or alternative embodiment, the method comprising orally administering to the subject a therapeutically effective amount of recombinant glucocerebrosidase (GCD) comprised in plant cells, wherein an amount in units of the GCD is up to 16 fold, e.g., 1-16 fold, e.g., 1-3 fold, 1.5-3 fold the amount in units of GCD administered by intravenous (I.V.) injection.

According to an additional or alternative embodiment, the method comprising orally administering to the subject a therapeutically effective amount of recombinant glucocerebrosidase (GCD) comprised in plant cells, wherein the administering is performed preprandially or over a light meal such that the stomach pH is above 2, 4 or 6 e.g., above 4. According to a specific embodiment, the pH is higher than 4, e.g., 4 -7.5 (saline pH).

Having a light meal (e.g., a glass of milk or a sandwich) prior to administering may be beneficial to elevate the stomach pH; and to activate the pancreatic enzymes. Alternatively, other means such as buffering agents can be sued to elevate the stomach pH above 2.

Heavy meals should be avoided to prevent enzymatic degradation by the pancreas in the upper intestine. In addition, substances that compromise the cell integrity prior to administration are avoided, e.g., juices or yogurts with enzymes that degrade cellulose

In this regard, according to a specific embodiment, the osmolarity of the oral formulation should be similar to physiological osmolarity (similar to saline) i.e., 250-300 mosM.

According to a specific embodiment, the therapeutically effective amount of GCD corresponds to 50-150 units/Kg/14 days.

According to a specific embodiment, the therapeutically effective amount of GCD corresponds to 50 units/Kg/14 days.

According to a specific embodiment, the therapeutically effective amount of GCD corresponds to 100 units/Kg/14 days.

According to a specific embodiment, the therapeutically effective amount of GCD corresponds to 150 units/Kg/14 days.

According to a specific embodiment, the therapeutically effective amount of GCD corresponds to 250-750 units/70 Kg/day.

According to a specific embodiment, the therapeutically effective amount of GCD corresponds to 250-500 units/70 Kg/day.

According to a specific embodiment, the therapeutically effective amount of GCD corresponds to 500-750 units/70 Kg/day.

According to a specific embodiment, the administering is effected daily.

As used herein “recombinant glucocerebrosidase (GCD) comprised in plant cells” refers to a genetically modified plant cell exogenously expressing GCD.

“Exogenously” refers to expression of a protein which is not native to the plant cell.

As used herein “glucocerebrosidase” or “GCD” refers to an enzyme with glucosylceramidase activity (EC 3.2.1.45) that is needed to cleave, by hydrolysis, the beta-glucosidic linkage of the chemical glucocerebroside, an intermediate in glycolipid metabolism.

According to a specific embodiment, the subject is human. The subject can be of any age including an infant, a child, a youngster and an adult. Thus, according to specific embodiments, the present teachings relate to the treatment of individuals weighing 0.6-200 Kg, 1-200 Kg, 3-150 Kg, 5-80 Kg, 50-80 Kg, 15-40 Kg, 3-14 Kg, or 3-110 Kg.

According to a specific embodiment, the glucocerebrosidase is the human enzyme, e.g., SEQ ID NO: 4 or 13.

As used herein the phrase “plant cells” refers to whole plants, portions thereof (e.g., leaf, root, fruit, seed) or cells isolated therefrom (homogeneous or heterogeneous populations of cells) which exogenously express the biologically active recombinant (exogenous) GCD.

As used herein the phrase “isolated plant cells” refers to plant cells which are derived from disintegrated plant cell tissue or plant cell cultures.

As used herein the phrase “plant cell culture” refers to any type of native (naturally occurring) plant cells, plant cell lines and genetically modified plant cells, which are not assembled to form a complete plant, such that at least one biological structure of a plant is not present. Optionally, the plant cell culture of this aspect of the present invention may comprise a particular type of a plant cell or a plurality of different types of plant cells. It should be noted that optionally plant cultures featuring a particular type of plant cell may be originally derived from a plurality of different types of such plant cells.

According to a specific embodiment, plant cells of the invention comprise an intact cell membrane and/or cell-wall, indicating that no deliberate destruction of these structures is needed prior to administration in order to deliver the enzyme. Thus, according to a specific embodiment, at least 30%, 40%, 50%, 60%, 70%, 80%, 90% or 100% cells administered comprise a substantially intact cell membrane and/or cell-wall.

Plant cells of the present invention are derived from a plant (or part thereof), preferably an edible and/or non toxic plant, which is amenable to genetic modification so as to express the recombinant protein therein.

Examples of plants which may be used in accordance with this aspect of the present invention include, but are not limited to, moss, algae, monocot or dicot, as well as other plants. Examples include, but are not limited to, leafy crops, oil crops, alfalfa, tobacco, tomatoes, bananas, carrots, lettuce, maize, cucumber, melon, potatoes, grapes and white clover.

The plant cell may optionally be any type of plant cell such as a plant root cell (i.e. a cell derived from, obtained from, or originally based upon, a plant root), more preferably a plant root cell selected from the group consisting of, a celery cell, a ginger cell, a horseradish cell and a carrot cell.

According to a specific embodiment, the plant cells are carrot cells.

According to a specific embodiment, the plant cells are tobacco cells.

According to a specific embodiment, the plant tobacco cells are BY-2 cells Or Nicotiana Benthamiana cells.

It will be appreciated that plant cell cultures originating from plant organ structures other than roots can be initiated, for example by transforming with Agrobacterium rhizogenes, and thereby inducing neoplastic structures known as hairy roots, that can be used for cultures (see, for example, U.S. Pat. No. 4,588,693 to Strobel et al), as further described hereinbelow. Thus, as described hereinabove, and detailed in the Examples section below, the plant root cell may be an Agrobacterium rhizogenes transformed root cell.

According to a specific embodiment, the plant cells are lyophilized plant cells.

In order to reach the lysosomes in the target cells, GCD is modified to include a terminally exposed mannose. WO2004/096978 and U.S. Pat. No. 7,951,557 teach constructs and methods for expressing biologically active GCD in plant cells (the teachings of which are herein incorporated by reference in their entirety). Thus, according to a specific embodiment, the GCD is linked at its N terminus to an endoplasmic reticulum signal peptide and at its C-terminus to a vacuolar signal peptide (see SEQ ID NO: 13 or 14 for example). According to a specific embodiment, the attachment of the signal peptides is directly to the amino acid sequence of GCD without the use of linkers.

According to a specific embodiment, the endoplasmic reticulum signal peptide is as set forth in SEQ ID NO: 1 or 12.

According to a specific embodiment, the vacuolar signal peptide is as set forth in SEQ ID NO: 2.

According to a specific embodiment, the main glycan structure of the glucocerebrosidase of the plant cells comprises at least one xylose residue and at least one exposed mannose residue, as measured by linkage analysis.

According to a specific embodiment, the glucocerebrosidase has an increased affinity for, and uptake into macrophages, in comparison with the corresponding affinity and uptake of a recombinant human glucocerebrosidase protein produced in mammalian cells, and having glucocerebrosidase catalytic activity.

Suspension cultures are preferably used in accordance with this aspect of the present invention, although callus cultures may also be used, as long as sterility is maintained.

Expression of the biologically active recombinant protein of this aspect of the present invention in cells of the above-described plant cell culture is effected by ligating a nucleic acid sequence expressing same (SEQ ID NO: 15) into a nucleic acid construct suitable for plant expression. In addition expression of the biologically active protein of this aspect of the present invention in cells of the above-described plant cell culture is effected by ligating a nucleic acid sequence driving the over expression of a plant gene.

Such a nucleic acid construct includes a cis-acting regulatory region such as a promoter sequence for directing transcription of the polynucleotide sequence in the cell in a constitutive or inducible manner. The promoter may be homologous or heterologous to the transformed plant/cell. Or alternatively, such a nucleic acid construct includes an enhancer/promoter element to be inserted into the plant genome in the vicinity to a plant gene (i.e., knock-in).

The promoter may be a plant promoter or a non-plant promoter which is capable of driving high levels of transcription of a linked sequence in the host cell, such as in plant cells and plants. The promoter may be either constitutive or inducible. For example, and not by way of limitation, an inducible promoter can be a promoter that promotes expression or increased expression of the lysosomal enzyme nucleotide sequence after mechanical gene activation (MGA) of the plant, plant tissue or plant cell.

Examples of constitutive plant promoters include, but are not limited to CaMV35S and CaMV19S promoters, FMV34S promoter, sugarcane bacilliform badnavirus promoter, CsVMV promoter, Arabidpsis ACT2/ACT8 actin promoter, Arabidpsis ubiquitin UBQ 1 promoter, barley leaf thionin BTH6 promoter, rice actin promoter, rbcS, the promoter for the chlorophyll a/b binding protein, AdhI, NOS and HMG2, or modifications or derivatives thereof.

An inducible promoter is a promoter induced by a specific stimulus such as stress conditions comprising, for example, light, temperature, chemicals, drought, high salinity, osmotic shock, oxidant conditions or in case of pathogenicity. Usually the promoter is induced before the plant is harvested and as such is referred to as a pre-harvest promoter. Examples of inducible pre-harvest promoters include, but are not limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr2O3J and str246C active in pathogenic stress.

The expression vectors used for transfecting or transforming the host cells of the invention can be additionally modified according to methods known to those skilled in the art to enhance or optimize heterologous gene expression in plants and plant cells. Such modifications include but are not limited to mutating DNA regulatory elements to increase promoter strength or to alter the protein of interest, as well as to optimizing codon usage. Construction of synthetic genes by altering the codon usage is described in for example PCT Patent Application 93/07278.

The nucleic acid construct can be, for example, a plasmid, a bacmid, a phagemid, a cosmid, a phage, a virus or an artificial chromosome. Preferably, the nucleic acid construct of the present invention is a plasmid vector, more preferably a binary vector.

The phrase “binary vector” refers to an expression vector which carries a modified T-region from Ti plasmid; enable to be multiplied both in E. coli and in Agrobacterium cells, and usually comprising reporter gene(s) for plant transformation between the two boarder regions. A binary vector suitable for the present invention includes pBI2113, pBI121, pGA482, pGAH, pBIG, pBI101 (Clonetech), pPI or modifications thereof.

It will be appreciated that production of active polypeptides in some cases comprises a sequence of events, commencing with expression of the polypeptide which may be followed by post translational modifications, e.g., glycosylation, dimeriztion, methylation and sulfhylation, hydroxylation.

Although plants are capable of glycosylating human proteins at the correct position, the composition of fully processed complex plant glycans differs from mammalian N-linked glycans. Plant glycans, do not have the terminal sialic acid residue or galactose residues common in animal glycans and often contain a xylose or fucose residue with a linkage that is generally not found in mammals (Jenkins et al., 14 Nature Biotech 975-981 (1996); Chrispeels and Faye in transgenic plants pp. 99-114 (Owen, M. and Pen, J. eds. Wiley & Sons, N.Y. 1996; Russell 240 Curr. Top. Microbio. Immunol. (1999).

The nucleic acid construct of the present invention can be utilized to stably or transiently transform plant cells. In stable transformation, the nucleic acid molecule of the present invention is integrated into the plant genome, and as such it represents a stable and inherited trait. In transient transformation, the nucleic acid molecule is expressed by the cell transformed but not integrated into the genome, and as such represents a transient expression of a specific protein.

There are various methods of introducing foreign genes into both monocotyledonous and dicotyledonous plants (Potrykus, I. (1991). Annu Rev Plant Physiol Plant Mol Biol 42, 205-225; Shimamoto, K. et al. (1989). Fertile transgenic rice plants regenerated from transformed protoplasts. Nature (1989) 338, 274-276).

The principal methods of the stable integration of exogenous DNA into plant genomic DNA include two main approaches:

(i) Agrobacterium-mediated gene transfer. See: Klee, H. J. et al. (1987). Annu Rev Plant Physiol 38, 467-486; Klee, H. J. and Rogers, S. G. (1989). Cell Culture and Somatic Cell Genetics of Plants, Vol. 6, Molecular Biology of Plant Nuclear Genes, pp.

2-25, J. Schell and L. K. Vasil, eds., Academic Publishers, San Diego, Calif.; and Gatenby, A. A. (1989). Regulation and Expression of Plant Genes in Microorganisms, pp. 93-112, Plant Biotechnology, S. Kung and C. J. Arntzen, eds., Butterworth Publishers, Boston, Mass. This is especially favored when root cells are used as host cells.

(ii) Direct DNA uptake. See, e.g.: Paszkowski, J. et al. (1989). Cell Culture and Somatic Cell Genetics of Plants, Vol. 6, Molecular Biology of Plant Nuclear Genes, pp. 52-68, J. Schell and L. K. Vasil, eds., Academic Publishers, San Diego, Calif.; and Toriyama, K. et al. (1988). Bio/Technol 6, 1072-1074 (methods for direct uptake of DNA into protoplasts). See also: Zhang et al. (1988). Plant Cell Rep 7, 379-384; and Fromm, M. E. et al. (1986). Stable transformation of maize after gene transfer by electroporation. Nature 319, 791-793 (DNA uptake induced by brief electric shock of plant cells). See also: Klein et al. (1988). Bio/Technology 6, 559-563; McCabe, D. E. et al. (1988). Stable transformation of soybean (Glycine max) by particle acceleration. Bio/Technology 6, 923-926; and Sanford, J. C. (1990). Biolistic plant transformation. Physiol Plant 79, 206-209 (DNA injection into plant cells or tissues by particle bombardment). See also: Neuhaus, J. M. et al. (1987). Theor Appl Genet 75, 30-36; and Neuhaus, J. M. and Spangenberg, G. C. (1990). Physiol Plant 79, 213-217 (use of micropipette systems). See U.S. Pat. No. 5,464,765 (glass fibers or silicon carbide whisker transformation of cell cultures, embryos or callus tissue). See also: DeWet, J. M. J. et al. (1985). “Exogenous gene transfer in maize (Zea mays) using DNA-treated pollen,” Experimental Manipulation of Ovule Tissue, G. P. Chapman et al., eds., Longman, New York-London, pp. 197-209; and Ohta, Y. (1986). High-Efficiency Genetic Transformation of Maize by a Mixture of Pollen and Exogenous DNA. Proc Natl Acad Sci U.S.A. 83, 715-719 (direct incubation of DNA with germinating pollen).

The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch, R. B. et al. (1988). “Leaf disc transformation.” Plant Molecular Biology Manual A5, 1-9, Kluwer Academic Publishers, Dordrecht). A supplementary approach employs the Agrobacterium delivery system in combination with vacuum infiltration. The Agrobacterium system is especially useful for in the creation of transgenic dicotyledonous plants.

There are various methods of direct DNA transfer into plant cells. In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

Although stable transformation is presently preferred, transient transformation of, for instance, leaf cells, meristematic cells, or the whole plant is also envisaged by the present invention. However, in this case measures are taken to exclude viral sequences or selection genes (e.g., antibiotic resistance) for regulatory purposes.

Transient transformation can be effected by any of the direct DNA transfer methods described above or by viral infection using modified plant viruses.

Viruses that have been shown to be useful for the transformation of plant hosts include cauliflower mosaic virus (CaMV), tobacco mosaic virus (TMV), and baculovirus (BV). Transformation of plants using plant viruses is described in, for example: U.S. Pat. No. 4,855,237 (bean golden mosaic virus, BGMV); EPA 67,553 (TMV); Japanese Published Application No. 63-14693 (TMV); EPA 194,809 (BV); EPA 278,667 (BV); and Gluzman, Y. et al. (1988). Communications in Molecular Biology: Viral Vectors, Cold Spring Harbor Laboratory, New York, pp. 172-189. The use of pseudovirus particles in expressing foreign DNA in many hosts, including plants, is described in WO 87/06261.

Construction of plant RNA viruses for the introduction and expression of non-viral exogenous nucleic acid sequences in plants is demonstrated by the above references as well as by: Dawson, W. O. et al. (1989). A tobacco mosaic virus-hybrid expresses and loses an added gene. Virology 172, 285-292; French, R. et al. (1986) Science 231, 1294-1297; and Takamatsu, N. et al. (1990). Production of enkephalin in tobacco protoplasts using tobacco mosaic virus RNA vector. FEBS Lett 269, 73-76.

If the transforming virus is a DNA virus, one skilled in the art may make suitable modifications to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of the DNA will produce the coat protein, which will encapsidate the viral DNA. If the virus is an RNA virus, the virus is generally cloned as a cDNA and inserted into a plasmid. The plasmid is then used to make all of the plant genetic constructs. The RNA virus is then transcribed from the viral sequence of the plasmid, followed by translation of the viral genes to produce the coat proteins which encapsidate the viral RNA.

Construction of plant RNA viruses for the introduction and expression in plants of non-viral exogenous nucleic acid sequences, such as those included in the construct of the present invention, is demonstrated in the above references as well as in U.S. Pat. No. 5,316,931.

In one embodiment, there is provided for insertion a plant viral nucleic acid, comprising a deletion of the native coat protein coding sequence from the viral nucleic acid, a non-native (foreign) plant viral coat protein coding sequence, and a non-native promoter, preferably the subgenomic promoter of the non-native coat protein coding sequence, and capable of expression in the plant host, packaging of the recombinant plant viral nucleic acid, and ensuring a systemic infection of the host by the recombinant plant viral nucleic acid. Alternatively, the native coat protein coding sequence may be made non-transcribable by insertion of the non-native nucleic acid sequence within it, such that a non-native protein is produced. The recombinant plant viral nucleic acid construct may contain one or more additional non-native subgenomic promoters. Each non-native subgenomic promoter is capable of transcribing or expressing adjacent genes or nucleic acid sequences in the plant host and incapable of recombination with each other and with native subgenomic promoters. In addition, the recombinant plant viral nucleic acid construct may contain one or more cis-acting regulatory elements, such as enhancers, which bind a trans-acting regulator and regulate the transcription of a coding sequence located downstream thereto. Non-native nucleic acid sequences may be inserted adjacent to the native plant viral subgenomic promoter or the native and non-native plant viral subgenomic promoters if more than one nucleic acid sequence is included. The non-native nucleic acid sequences are transcribed or expressed in the host plant under control of the subgenomic promoter(s) to produce the desired products.

In a second embodiment, a recombinant plant viral nucleic acid construct is provided as in the first embodiment except that the native coat protein coding sequence is placed adjacent to one of the non-native coat protein subgenomic promoters instead of adjacent to a non-native coat protein coding sequence.

In a third embodiment, a recombinant plant viral nucleic acid construct is provided comprising a native coat protein gene placed adjacent to its subgenomic promoter and one or more non-native subgenomic promoters inserted into the viral nucleic acid construct. The inserted non-native subgenomic promoters are capable of transcribing or expressing adjacent genes in a plant host and are incapable of recombination with each other and with native subgenomic promoters. Non-native nucleic acid sequences may be inserted adjacent to the non-native subgenomic plant viral promoters such that the sequences are transcribed or expressed in the host plant under control of the subgenomic promoters to produce the desired product.

In a fourth embodiment, a recombinant plant viral nucleic acid construct is provided as in the third embodiment except that the native coat protein coding sequence is replaced by a non-native coat protein coding sequence.

Viral vectors are encapsidated by expressed coat proteins encoded by recombinant plant viral nucleic acid constructs as described hereinabove, to produce a recombinant plant virus. The recombinant plant viral nucleic acid construct or recombinant plant virus is used to infect appropriate host plants. The recombinant plant viral nucleic acid construct is capable of replication in a host, systemic spread within the host, and transcription or expression of one or more foreign genes (isolated nucleic acid) in the host to produce the desired protein.

In another embodiment, the transformation vehicle comprises viral derived sequences comprising RNA dependent RNA polymerase (RdRp), subgenomic promoter and/or a partial or complete movement protein sequences wherein all the above nucleic acid fragments are cloned into a binary vector. (Gleba et al, Current Opinion in Plant Biology 2004, 7:182-188). In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced into the cells preferably via particle bombardment, with the aim of introducing at least one exogenous nucleic acid molecule into the chloroplasts. The exogenous nucleic acid is selected by one ordinarily skilled in the art to be capable of integration into the chloroplast's genome via homologous recombination, which is readily effected by enzymes inherent to the chloroplast. To this end, the exogenous nucleic acid comprises, in addition to a gene of interest, at least one nucleic acid sequence derived from the chloroplast's genome. In addition, the exogenous nucleic acid comprises a selectable marker, which by sequential selection procedures serves to allow an artisan to ascertain that all or substantially all copies of the chloroplast genome following such selection include the exogenous nucleic acid. Further details relating to this technique are found in U.S. Pat. Nos. 4,945,050 and 5,693,507, which are incorporated herein by reference. A polypeptide can thus be produced by the protein expression system of the chloroplast and become integrated into the chloroplast's inner membrane.

Regardless of the method employed, following transformation, plant propagation occurs. In this case micropropagation is effected to include initial tissue culturing; and tissue culture multiplication to obtain enough cells for further use.

Methods of plant cell culturing are well known in the art. Culturing conditions (e.g., culture medium, temperature, gas environment, and bioreactor) may be adjusted according to the plant cell used and the expressed protein to achieve optimal expression. Typically, culturing is effected under standard plant cell culture conditions using any conventional plant culture medium. It will be appreciated that plant culture medium includes both aqueous media and dry and concentrated media to which water can be added to produce aqueous media for culturing plant cells (see e.g., U.S. Pat. Nos. 6,020,169 and 6,589,765).

Examples of plant culture media, which can be used in accordance with the present invention, include, but not limited to, the following well known media:

Anderson (Anderson, In Vitro 14:334, 1978; Anderson, Act. Hort., 112:13, 1980), Chee and Pool (Sci. Hort. 32:85, 1987), CLC/Ipomoea (CP) (Chee et al., J. Am. Soc. Hort. Sci. 117:663, 1992), Chu (N.sub.6) (Chu et al., Scientia Sinic. 18:659, 1975; Chu, Proc. Symp. Plant Tiss. Cult., Peking 43, 1978), DCR (Gupta and Durzan, Plant Cell Rep. 4:177, 1985), DKW/Juglans (Driver and Kuniyuki, HortScience 19:507, 1984; McGranahan et al., in: Bonga and Durzan, eds., Cell and Tissue Culture in Forestry, Martinus Nijhoff, Dordrecht, 1987), De Greef and Jacobs (De Greef and Jacobs, Plant Sci. Lett. 17:55, 1979), Eriksson (E R) (Eriksson, Physiol. Plant. 18:976, 1965), Gamborg's B-5 (Gamborg et al., Exp. Cell Res. 50:151, 1968), Gresshoff and Doy (DBM2) (Gresshoff and Doy, Z Pflanzenphysiol. 73:132, 1974), Heller (Heller, Ann. Sci. Nat. Bot. Biol. Veg. 11th Ser. 14:1, 1953), Hoagland's (Hoagland and Amon, Circular 347, Calif. Agr. Exp. Stat., Berkeley, 1950), Kao and Michayluk (Kao and Michayluk, Planta 126:105, 1975), Linsmaier and Skoog (Linsmaier and Skoog, Physiol. Plant. 18:100, 1965), Litvay's (L M) (Litvay et al., Plant Cell Rep. 4:325, 1985), McCown's Woody Plant medium (Lloyd and McCown, Proc. Int. Plant Prop. Soc. 30:421, 1981), Murashige and Skoog and various well-known modifications thereof (Murashige and Skoog, Physiol. Plant. 15:473, 1962), Nitsch and Nitsch (Nitsch and Nitsch, Science 163:85, 1969), Quoirin and Lepoivre (Quoirin et al., C. R. Res. Sta. Cult. Fruit Mar., Gembloux 93, 1977), Schenk and Hildebrandt (Schenk and Hildebrandt, Can. J. Bot. 50:199, 1972), White's (White, The Cultivation of Animal and Plant Cells, Ronald Press, NY, 1963), etc. A number of such plant culture media are commercially available from Sigma (St. Louis, Mo.) and other vendors as dry (powdered) media and dry basal salts mixtures, for example.

Preferably, culturing is effected using the high yield disposable plant culture device, which has been shown to be effective for the production of biologically active peptides and polypeptides in culture (see WO98/13469 and WO08/135991, which are incorporated herein by reference in their entirety).

According to a specific embodiment, once plant cells expressing the above-described recombinant protein are obtained, they are lyophilized, although the use of fresh (non-lyophilized cells) is also contemplated herein.

Prior to lyophilization the cells may be washed to remove any cell debris that may be present in the growth medium.

As the cells are being prepared for lyophilization, it is sometimes desirable to incubate the cells in a maintenance medium to reduce the metabolic processes of the cells.

Pretreatment (although not necessary) can be performed at room temperature or at temperatures in which the plant cells are typically cultured. Pretreatment is performed at about room temperature (20° C.) for ease of handling and as most plant cells are fairly stable at room temperature. Stabilizers can be added directly to the medium and replenished as necessary during the pretreatment process.

Pretreatments may also involve incubating cells in the presence of one or more osmotic agents. Examples of useful osmotic agents include sugars such as saccharides and saccharide derivatives, amino or imino acids such as proline and proline derivatives, or combinations of these agents. Some of the more useful sugars and sugar derivatives are fructose, glucose, maltose, mannitol, sorbitol, sucrose and trehalose. Osmotic agents are utilized at a concentration that prepares cells for subsequent lyophilization.

Lyophilization is directed at reducing the water content of the cells by vacuum evaporation. Vacuum evaporation involves placing the cells in an environment with reduced air pressure. Depending on the rate of water removal desired, the reduced ambient pressure operating at temperatures of between about −30° C. to −50° C. may be at 100 ton, 1 ton, 0.01 ton or less. According to a specific embodiment, the cells are lyophilized by freezing to -40° C. and then applying a vacuum to a pressure of 0.1 mbar for overnight. The cells are then heated to −10° C. so all the ice content will be sublimated and evaporated. Under conditions of reduced pressure, the rate of water evaporation is increased such that up to 60-95% of the water in a cell can be removed.

According to a specific embodiment, lyophilization removes over 60%, 70%, 80% or specifically over 90%, 91%, 92%, 93%, 94%, 95% or 98% of the water from the cells. According to a specific embodiment, the final water content is about 5-10%, 5-8% or 6-7%.

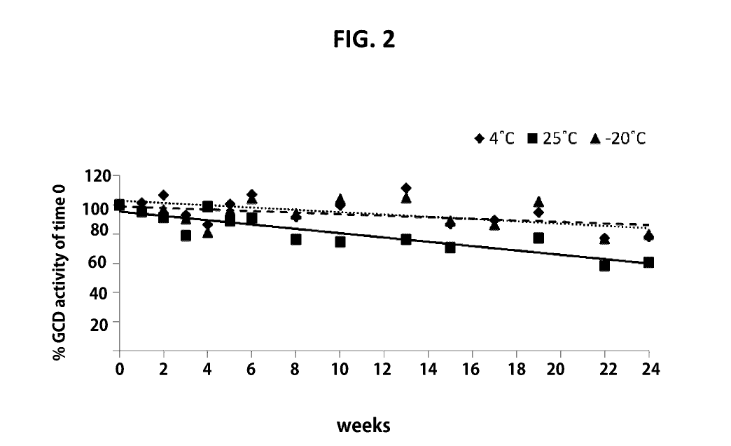

A specific lyophilization protocol is provided in the Examples section which follows. As shown in FIG. 2, prGCD in lyophilized carrot cells maintain substantial activity over months (about 6 months) at room temperature (25° C., at least 70% of the activity at time zero).

The present inventors were able for the first time to determine the bioavailability factor of orally administered GCD which is comprised in plant cells. See Example 10 of the Examples section which follows.

Thus, according to an aspect of the invention, there is provided a method of determining relative bioavailability of orally administered GCD comprised in plant cells.

The method comprising measuring a pharmacokinetic factor or a pharmacodynamic factor:

(i) of orally administered GCD comprised in plant cells;

(ii) of intravenously administered soluble GCD,

wherein a ratio (i) and (ii) is indicative of the relative bioavailability of orally administered GCD comprised in plant cells.

“Bioavailability” refers to the rate and extent of drug input into the systemic circulation measured as the fraction or percent of the administered dose that absorbs intact and maintains activity.

“Relative bioavailability (F)” measures the bioavailability (estimated as the AUC) of oral GCD comprised in plant cells when compared to soluble GCD injected intravenously.

Bioavailability can be measured by determining a pharmacokinetic or pharmacodynamic factor. According to a specific embodiment the bioavailability is determined as enzymes activity in serum or blood leukocytes.

According to a specific embodiment, the bioavailability is determined in animal subjects such as rats and pigs that are administered with the formulation.

The bioavailability or relative bioavailability can also be determined in human subjects such as Gaucher's disease patients. Accordingly, the present teachings can be used to personally determine the optimal dose of orally administered GCD comprised in plant cells in a human subject that is treated with injectable GCD e.g., imiglucerase (Genzyme Inc.) velaglucerase alfa (Shire Inc.) or taliglucerase alfa (Protalix Ltd.).

Thus, according to an aspect there is provided a method of treating or designing a treatment regimen for a subject having Gaucher's disease, the method comprising:

(a) determining relative bioavailability of orally administered GCD comprised in plant cells in the subject (as described above); and

(b) designing an oral treatment regimen for the subject according to the bioavailability (F).

Alternatively or additionally, there is provided a method of personalized therapy of a subject having Gaucher's disease, the method comprising determining the therapeutic effective amount of intravenously administered soluble GCD in the subject and designing a treatment regimen for orally administered GCD in the subject based on the therapeutic effective amount multiplied by up to 16, e.g., 1-16, 4-16, 1-3.

Based on these teachings the present inventors have uncovered the relative bioavailability of orally administered GCD comprised in plant cells. The present inventors have realized through laborious experimentation that the relative bioavailability of orally administered GCD comprised in plant cells is up to 16, e.g., 1-16, fold higher than the amount in units of GCD administered by intravenous (I.V.) injection.

As mentioned, that the inclusion of GCD in plant cells and its oral administration results in a slow-release-like effect, whereby the enzyme is slowly released to the circulation (digestion-dependent), thus maintaining essentially constant levels of the enzyme in the blood. High frequency of administration (relative to the i.v. route) ensures maintenance of high levels of enzymes in the circulation. Thus, the administration of the enzyme in the plant cells, high frequency administration or the combination of same may allow reducing the overall dose of the enzyme administered (again, in comparison to the i.v. administered doses).

Thus, according to a specific embodiment, for oral administration, the relative bioavailability as defined herein is 1-3, 1.5-5, 1-16, 1.5-16, 2-16, 3-16, 4-16, 4-12, 6-15, 6-12, 8-12, 9-11 or specifically 10 fold higher than for i.v. injection.

The dose for i.v. treatment is typically 30-60 units/kg/14 days the dose is adjusted in the course of treatment in the range of 10-120 units/kg/14 days.

Table 1 below provides non-limiting examples of unit doses expressed in units/kg/14 days.

TABLE 1

| | Oral | Oral | Oral | Oral |
|---|---|---|---|---|
| I.V. | F = 1-16 | F = 4-16 | F = 10 | F = 8-12 |
| 30-60 | 30-960 | 120-960 | 300-600 | 240-720 |
| 10-120 | 10-1920 | 40-1920 | 100-1200 | 80-1440 |

According to some embodiments of the invention, the administering is effected at a dose of 40-1920 units/Kg.

According to some embodiments of the invention, the administering is effected at a dose of 100-1200 units/Kg.

According to some embodiments of the invention, the administering is effected at a dose of 600-1200 units/Kg.

According to some embodiments of the invention, the administering is effected at a dose of 100-1200 units/Kg.

According to some embodiments of the invention, the administering is effected at a dose of 120-960 units/Kg.

According to some embodiments of the invention, the administering is effected at a dose of 300-600 units/Kg.

According to some embodiments of the invention, the administering is effected at a dose of 1-1000 units/Kg.

According to some embodiments of the invention, the administering is effected at a dose of 1-500 units/Kg.

According to some embodiments of the invention, the administering is effected at a dose of 1-400 units/Kg.

According to some embodiments of the invention, the administering is effected at a dose of 1-300 units/Kg.

According to some embodiments of the invention, the administering is effected at a dose of 1-200 units/Kg.

According to some embodiments of the invention, the administering is effected at a dose of 50-150 units/Kg.

According to some embodiments of the invention, the administering is effected at a dose of 50-100 units/Kg.

According to some embodiments of the invention, the administering is effected at a dose of 100-150 units/Kg.

According to some embodiments of the invention, the administering is effected at a dose of 1-100 units/Kg.

According to some embodiments of the invention, the administering is effected at a dose of 1-80 units/Kg.

According to some embodiments of the invention, the administering is effected at a dose of 1-60 units/Kg.

According to some embodiments of the invention, the administering is effected at a dose of 1-50 units/Kg.

According to some embodiments of the invention, the administering is effected at a dose of 1-40 units/Kg.

According to some embodiments of the invention, the administering is effected at a dose of 1-30 units/Kg.

According to some embodiments of the invention, the administering is effected at a dose of 1-20 units/Kg.

According to some embodiments of the invention, the administering is effected at a dose of 1-10 units/Kg.

As used herein the term unit refers to the amount of GCD that catalyzes the hydrolysis of one micromole of the synthetic substrate para-nitrophenyl-beta-D-glucopyranoside (pNP-Glc) per minute at 37° C.

Since the mode of administration is oral, the administration can be effected daily by dividing the above doses by 14 or more.

According to a specific embodiment, administering is effected daily, i.e., every day.

According to a further specific embodiment, administering is effected once daily.

According to a further specific embodiment, administering is effected twice daily.

According to a further specific embodiment, administering is effected daily, three times a day.

According to a further specific embodiment, administering is effected daily, four times a day.

According to a specific embodiment, administering is effected every two days.

According to a further specific embodiment, administering is effected once every two days.

According to a further specific embodiment, administering is effected twice every two days.

According to a further specific embodiment, administering is effected every two days, three times a day.

According to a further specific embodiment, administering is effected every two days, four times a day.

Alternatively administering is effected twice a week (once, twice, thrice or four times a day).

In some embodiments, it is desirable to minimize the volume of cells ingested at each administration, and thus the dosage is divided into small volume doses administered at higher frequency. For example, the composition of GCD in cells can be prepared in a single volume of, for example, 500 ml, or, alternatively, the same dose of GCD in cells can be prepared in 2, or 3 or 4 or 5 portions of the dose, each having a volume of 250, 333, 125 or 100 ml, respectively, to be administered at two, three, four or five times during the day, respectively. Thus, volumes of the dosage can vary according to the individual requirements of the treatment regimen and of the patient's needs and preferences.

Other modes of administration are also contemplated. The total amount of the enzyme realized for two weeks is divided according to the desired regimen.

It will be appreciated that treatment may be adjusted according to clinical manifestation i.e., severity of the disease. The skilled artisan would know how to determine clinical manifestation of Gaucher's disease (enzymatic activity in the plasma, liver size etc.).

Further, it will be appreciated that, for oral administration of GCD in plant cells, the integrity of the subject's gastrointestinal tract can be a significant factor in determining the dosage. Thus, the dosage and/or dosage regimen and/or composition of the invention can be adjusted according to gastrointestinal health factors such as food allergies, GI inflammatory disorders, and the like. For example, sensitive individuals may receive smaller doses, more frequently administered, or administered in an alternative formulation, than individuals exhibiting no GI sensitivity. The skilled artisan would know how to determine clinical manifestation of gastrointestinal disease or disorder (constipation, diarrhea, etc.).

According to a specific embodiment the subject is selected not manifesting a GI disorder which is not directly associated with Gaucher's disease. The GI disorder can be in any portion of the gastrointestinal tract which affects absorption. Examples of such GI disorders include but are not limited to inflammatory gastrointestinal disorders, functional gastrointestinal disorders, infectious (e.g. viral, bacterial, parasitic) gastrointestinal disorders, gastrointestinal cancer (primary or secondary) or a combination of gastrointestinal disorders. Examples of an inflammatory gastrointestinal disorder include, but are not limited to, ulcerative colitis, Crohn's disease or a combination thereof. An example of a functional gastrointestinal disorder includes, but is not limited to, irritable bowel disease. Examples of infectious gastrointestinal disorders include, but are not limited to viral gastroenteritis, amoebiasis, giardia, tapeworm, ascaris, etc.

Table 2 below provides non-limiting examples for unit doses for oral administration once daily (units/kg/day).

TABLE 2

| | Oral | Oral | Oral | Oral |
|---|---|---|---|---|
| Daily i.v. | F = 1-16 | F = 4-16 | F = 10 | F = 8-12 |
| 30-60/14 | 2-69 | 8-69 | 21-43 | 17-52 |
| 10-120/14 | 0.5-138 | 2-138 | 7-86 | 5-103 |

Administration can be effected such as with every meal. The administration can be done every two days in which case the preceding numbers are multiplied by two.

The subject's disease stability is monitored following switch. Monitoring can include any of the above mentioned clinical and biological parameters such as liver volume, spleen volume and complete blood count. If needed, the subject may be prescribed again ERT in case test results show deterioration in disease symptoms.

The cells expressing the recombinant GCD (e.g., powder which comprises the lyophilized plant cells) can be packed in a unit dosage form formulated as an oral nutritional form or as a pharmaceutical composition. It will be appreciated that in the latter, the dosage form is mainly intended for use for children (due to volume constraints).

Thus, according to an aspect of the invention there is provided a unit dosage form comprising 1-11,000 units recombinant GCD comprised in plant cells. It will be appreciated that this range is aimed at a minimal daily dose administered four times a day to maximal daily dose (once a day) in patients weighing from 2-75 Kg.

According to an embodiment the unit dosage form comprises 4-11000 units recombinant GCD comprised in plant cells.

According to an embodiment the unit dosage form comprises 14-6450 units recombinant GCD comprised in plant cells.

According to an embodiment the unit dosage form comprises 10-5175 units recombinant GCD comprised in plant cells.

According to an embodiment the unit dosage form comprises 32-5175 units recombinant GCD comprised in plant cells.

According to an embodiment the unit dosage form comprises 42-3225 units recombinant GCD comprised in plant cells.

According to an embodiment the unit dosage form comprises 34-3900 units recombinant GCD comprised in plant cells.

According to an embodiment the unit dosage form comprises 214-11000 units recombinant GCD comprised in plant cells.

According to an embodiment the unit dosage form comprises 525-6450 units recombinant GCD comprised in plant cells.

According to an embodiment the unit dosage form comprises 375-7725 units recombinant GCD comprised in plant cells.

According to an embodiment the unit dosage form comprises 600-5175 units recombinant GCD comprised in plant cells.

According to an embodiment the unit dosage form comprises 1575-3325 units recombinant GCD comprised in plant cells.

According to an embodiment the unit dosage form comprises 1275-3900 units recombinant GCD comprised in plant cells.

According to some embodiments of the invention, the unit dosage form comprises 1-3000 units recombinant GCD comprised in plant cells.

According to some embodiments of the invention, the unit dosage form comprises 700-1500 units recombinant GCD comprised in plant cells.

According to some embodiments of the invention, the unit dosage form comprises 1-2000 units recombinant GCD comprised in plant cells.

According to some embodiments of the invention, the unit dosage form comprises 1-1000 units recombinant GCD comprised in plant cells.

According to some embodiments of the invention, the unit dosage form comprises 1-500 units recombinant GCD comprised in plant cells.

According to some embodiments of the invention, the unit dosage form comprises 1-100 units recombinant GCD comprised in plant cells.

According to some embodiments of the invention, the unit dosage form comprises 250-750 units recombinant GCD comprised in plant cells.

According to some embodiments of the invention, the unit dosage form comprises 250-500 units recombinant GCD comprised in plant cells.

According to some embodiments of the invention, the unit dosage form comprises 500-750 units recombinant GCD comprised in plant cells.

According to some embodiments of the invention, the unit dosage form comprises 500 units recombinant GCD comprised in plant cells.

According to some embodiments of the invention, the unit dosage form comprises 750 units recombinant GCD comprised in plant cells.

According to some embodiments of the invention, the unit dosage form comprises 250 units recombinant GCD comprised in plant cells.

It will be appreciated that these numbers may be multiplied or divided if administering is effected at lower frequencies (e.g., every 2-3 days) or administering is effected more than once a day (e.g., two, three or four times a day).

The cells may be formulated as a solid, formulated as a liquid or formulated as a powder. In some embodiments, the cells are resuspended, lyophilized cells.

Thus, the oral dosage form may be provided as an oral nutritional form (e.g., as long as the protein is not exposed to denaturing conditions which include heating above 37° C. and compression), as a complete meal, as a powder for dissolution, e.g. health drinks, as a solution, as a ready-made drink, optionally low calorie, such as a soft drink, including juices, milk-shake, yoghurt drink, smoothie or soy-based drink, in a bar, or dispersed in foods of any sort, such as baked products, cereal bars, dairy bars, snack-foods, breakfast cereals, muesli, candies, tabs, cookies, biscuits, crackers (such as a rice crackers), chocolate, and dairy products.

Table 3 below provides the different consistencies reached with 10 gr. of lyophilized cells. The skilled artisan will know how to employ the below values with the desired dose of enzyme and corresponding amount of cells.

TABLE 3

| Lyophilized cells:Volume of liquid (parts) | Consistency |
|---|---|
| 1:0.5 | Hard dough |
| 1:1 | Soft dough |
| 1:5 | Purée |
| 1:7.5 | Yogurt |
| 1:15 | Shake |
| 1:22.5 | Fruit (opaque) juice |

Alternatively, cells of the present invention can be administered to the subject in a pharmaceutical composition where they are mixed with suitable carriers or excipients.

As used herein, a “pharmaceutical composition” refers to a preparation of cells expressing GCD with other chemical components such as physiologically suitable carriers and excipients. The purpose of a pharmaceutical composition is to facilitate administration of a compound to an organism.

As used herein, the term “active ingredient” refers to the cells expressing GCD accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases. Preferably the carrier used is a non-immunogenic carrier and further preferably does not stimulate the gut associated lymphatic tissue.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added.

The dosage forms may include additives such as one or more of calcium, magnesium, iron, zinc, phosphorus, vitamin D and vitamin K. A suitable daily amount is 0.1 mg to 3.6 g calcium, preferably 320 to 530 mg. In general, the daily dosage of vitamins and minerals in the nutritional formulation or medicament of the invention is 25-100% by weight of the dosages recommended by the health authorities. Dietary fiber may also be a component of the compositions of the invention. Further components of the supplement may include any bioactive compounds or extracts which are known to have health benefits, especially for improving physical performance.

Generally the unit dosage form may further comprise an antioxidant (exemplary embodiments are provided above-. In another embodiment, the antioxidant is a pharmaceutically acceptable antioxidant. In another embodiment, the antioxidant is selected from the group consisting of vitamin E, superoxide dismutase (SOD), omega-3, and beta-carotene.

In another embodiment, the unit dosage form further comprises an enhancer of the biologically active protein or peptide. In another embodiment, the unit dosage form further comprises a cofactor of the biologically active protein or peptide.

In another embodiment, a unit dosage form of the present invention further comprises pharmaceutical-grade surfactant. Surfactants are well known in the art, and are described, inter alia, in the Handbook of Pharmaceutical Excipients (eds. Raymond C Rowe, Paul J Sheskey, and Sian C Owen, copyright Pharmaceutical Press, 2005). In another embodiment, the surfactant is any other surfactant known in the art.

In another embodiment, a unit dosage form of the present invention further comprises pharmaceutical-grade emulsifier or emulgator (emollient). Emulsifiers and emulgators are well known in the art, and are described, inter alia, in the Handbook of Pharmaceutical Excipients (ibid). Non-limiting examples of emulsifiers and emulgators are eumulgin, Eumulgin B1 PH, Eumulgin B2 PH, hydrogenated castor oil cetostearyl alcohol, and cetyl alcohol. In another embodiment, the emulsifier or emulgator is any other emulsifier or emulgator known in the art.

In another embodiment, a unit dosage form of the present invention further comprises pharmaceutical-grade stabilizer. Stabilizers are well known in the art, and are described, inter alia, in the Handbook of Pharmaceutical Excipients (ibid). In another embodiment, the stabilizer is any other stabilizer known in the art.

In another embodiment, a unit dosage form of the present invention further comprises an amino acid selected from the group consisting of arginine, lysine, aspartate, glutamate, and histidine. In another embodiment, analogues and modified versions of arginine, lysine, aspartate, glutamate and histidine are included in the terms “arginine,” “lysine,” “aspartate”, “glutamate” and “histidine,” respectively. In another embodiment, the amino acid provides additional protection of ribonuclease or other active molecules. In another embodiment, the amino acid promotes interaction of biologically active protein or peptide with a target cell. In another embodiment, the amino acid is contained in an oil component of the unit dosage form.

In another embodiment, a unit dosage form of the present invention further comprises one or more pharmaceutically acceptable excipients, into which the matrix carrier unit dosage form is mixed. In another embodiment, the excipients include one or more additional polysaccharides. In another embodiment, the excipients include one or more waxes. In another embodiment, the excipients provide a desired taste to the unit dosage form. In another embodiment, the excipients influence the drug consistency, and the final dosage form such as a gel capsule or a hard gelatin capsule.

Non limiting examples of excipients include: Antifoaming agents (dimethicone, simethicone); Antimicrobial preservatives (benzalkonium chloride, benzelthonium chloride, butylparaben, cetylpyridinium chloride, chlorobutanol, chlorocresol, cresol, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); Chelating agents (edetate disodium, ethylenediaminetetraacetic acid and salts, edetic acid); Coating agents (sodium carboxymethyl-cellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcrystalline wax, zein); Colorants (caramel, red, yellow, black or blends, ferric oxide); Complexing agents (ethylenediaminetetraacetic acid and salts (EDTA), edetic acid, gentisic acid ethanolmaide, oxyquinoline sulfate); Desiccants (calcium chloride, calcium sulfate, silicon dioxide); Emulsifying and/or solubilizing agents (acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate, stearic acid, trolamine, emulsifying wax); Flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); Humectants (glycerin, hexylene glycol, propylene glycol, sorbitol); Polymers (e.g., cellulose acetate, alkyl celluloses, hydroxyalkylcelluloses, acrylic polymers and copolymers); Suspending and/or viscosity-increasing agents (acacia, agar, alginic acid, aluminum monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum); Sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); This list is not meant to be exclusive, but instead merely representative of the classes of excipients and the particular excipients which may be used in oral dosage unit dosage forms of the present invention.

Conventional additives may be included in the compositions of the invention, including any of those selected from preservatives, chelating agents, effervescing agents, natural or artificial sweeteners, flavoring agents, coloring agents, taste masking agents, acidulants, emulsifiers, thickening agents, suspending agents, dispersing or wetting agents, antioxidants, and the like. Flavoring agents can be added to the compositions of the invention to aid in compliance with a dosing regimen. Typical flavoring agents include, but are not limited to natural or synthetic essences, oils and/or extracts of orange, lemon, mint, berry, chocolate, vanilla, melon and pineapple. In some embodiments the compositions are flavored with pineapple flavoring or a combination of flavorings (e.g. pineapple and grape). For example, a dose is given in mineral water; including Pineapple flavored diet syrup, Prigat (7 ml/100 ml water) and Concentrated grape flavor (Frutarom) 2 drops/100 ml.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Plasma Levels of Glucosylceramide Levels Following Enzyme Administration Via Bolus I.V. Injection or Via Daily Oral Administration

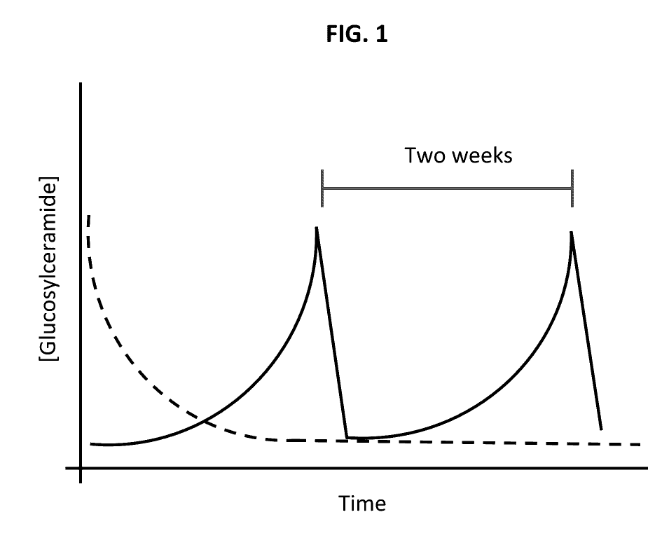

Current treatment of Gaucher disease is based on intravenous (i.v.) bolus injection every two weeks. FIG. 1 shows the theoretical assumption of the effect of such an administration mode on the accumulation of the GCD substrate (glucosylceramide) during two weeks. Following administration the levels of the substrate are brought down to the basic level. Without being bound to theory, oral administration optimally allows a daily treatment that keeps the substrate to its basic level. It is contemplated that less units can achieve a therapeutic effect when given in a daily dose in a manner where the enzyme is released from the cells to the GIT and is then absorbed to the circulation in a continuous manner as opposed to a pulse like administration manner, so all enzyme that reaches target organs will be exposed to its substrate.

### Example 2

### Lyophilized Plant Cells Maintain Substantial Activity of Plant Recombinant GCD (prGCD) Expressed Therein Over Months at Room Temperature

Expression of prGCD in carrot cells is described in details in WO2008/132743 which is hereby incorporated by reference in its entirety.

The cells were lyophilized by freezing to −40° C. Vacuum was applied to a pressure of 0.1 mbar overnight. The cells were heated to −10° C. for 72 hours and then to 20° C. Upon termination of the lyophilization process, the water content was 6.7%. The cells were then weighed into small aliquots that were kept under a humidity control for 24 weeks at room temperature, 4° C. or 25° C. At each time point, the cells were removed from the desiccators, reconstituted with 10×W/V extraction buffer (0.125% sodium taurocholate; 60 mM phosphate citrate buffer pH 6.0; 0.15% Triton-X100; pH 5.5) and the proteins were extracted using a TissueLyser (Retsch MM400; Haan, Germany). The extracts were then tested for GCD activity by the calorimetric method using the artificial substrate p-nitrophenyl-β-D-glucopyranoside (PNP)(catalog number N7006, Sigma-aldrich).

Results

Lyophilized carrot cells expressing prGCD were maintained in a desicator (−20° C., 4° C. or 25° C.). The recombinant protein was extracted from the cells and tested for its activity. As shown in FIG. 2, prGCD in lyophilized carrot cells maintains substantial activity over months at room temperature, 4° C. or −20° C.

### Example 3

### prGCD can Cross the Epithelial Barrier in an In-Vitro Model

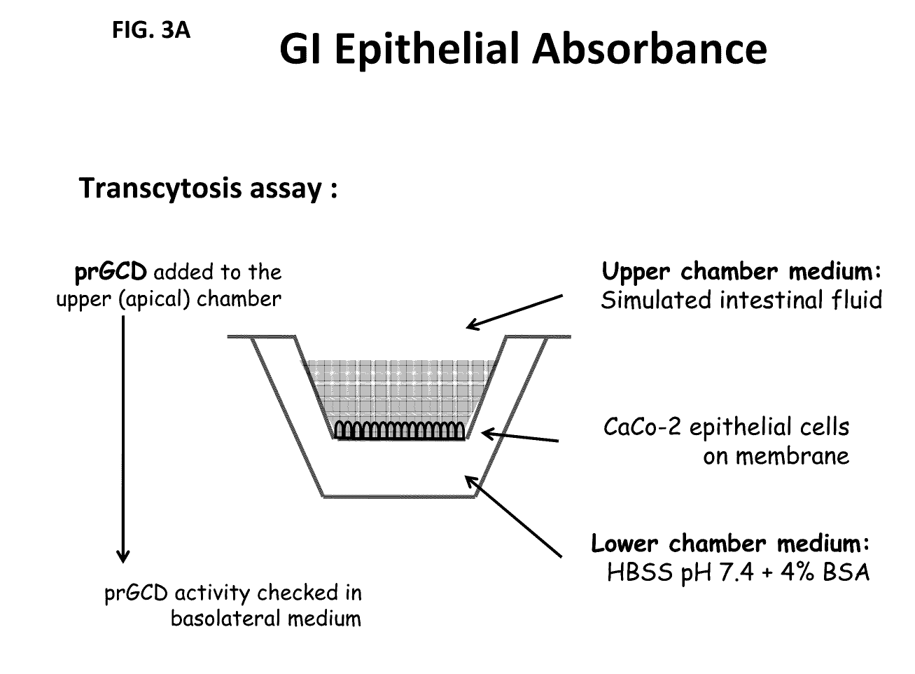

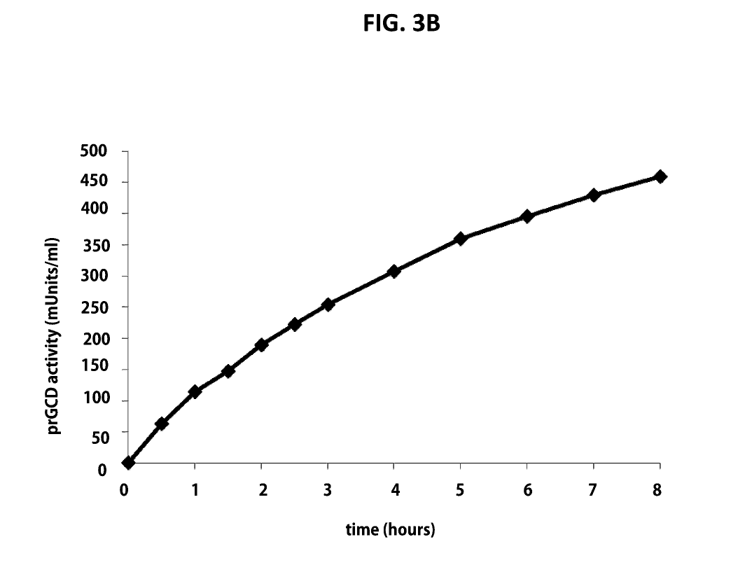

The ability of prGCD to cross the epithelial barrier was tested in an in-vitro Caco2 model (described in FIG. 3A, for epithelial absorbance). Transcytosis of GCD was performed in triplicate using three independent monolayers as described previously (Tzaban et al., 2009, J Cell Biol. 185(4):673-84). In brief, cells were washed with Hank's buffer salts solution (HBSS) containing 10 mM Hepes, pH 7.4, and then incubated with HBSS simulating the intestinal fluid in a fasted state at pH 6.0, for 10 min. prGCD was added at the apical chamber for a continuous uptake at 37° C. The medium in the basolateral chamber was collected after the indicated time points and prGCD activity was tested as described above using the calorimetric method. Apparent permeability coefficient (Papp) calculation formula is provided below:

$$Papp \frac{d Q}{d t} * \frac{1}{AC} Or$$

$$\begin{matrix}Papp = \frac{\begin{matrix}( slope of \frac{activity}{initial concentration} / time ( sec ) ) * \\ basolateral volume ( ml )\end{matrix}}{insert area ( {cm}^{2} )} \\ = \frac{\frac{1}{sec} * {cm}^{3}}{{cm}^{2}} = \frac{cm}{sec}\end{matrix}$$

Results

prGCD was added to an apical chamber in stimulated intestinal medium at 6.8 units/ml. Transcytosis was measured at the basolateral medium after the indicated times at 37° C. The rising activity at the basolateral side indicates that prGCD can cross the epithelial barrier with Papp of 1.39*10⁻⁷ cm/sec (FIG. 3B).

### Example 4

### Timeline of Carrot Cells Passing Through the Stomach

Three rats per group were gavage fed with carrot cells expressing prGCD. Each group was sacrificed at different time points post feeding from 1-24 hours. The content of the GIT was collected and tested for total content weight and prGCD activity. Plasma and liver were also tested for their GCD activity.

Results

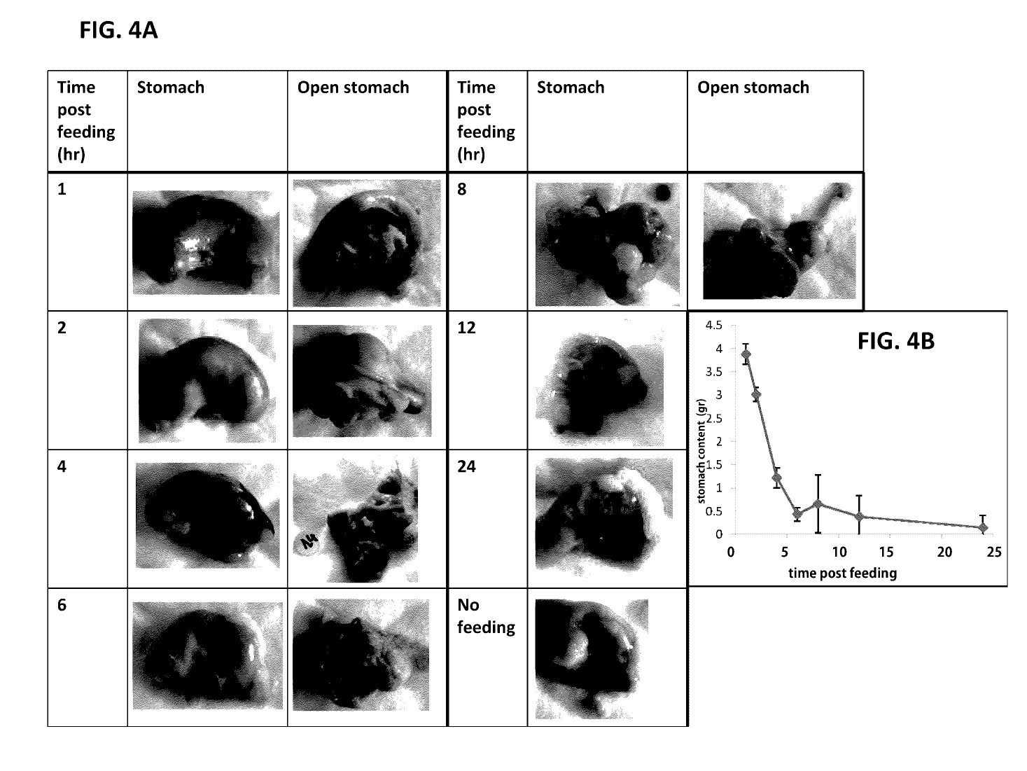

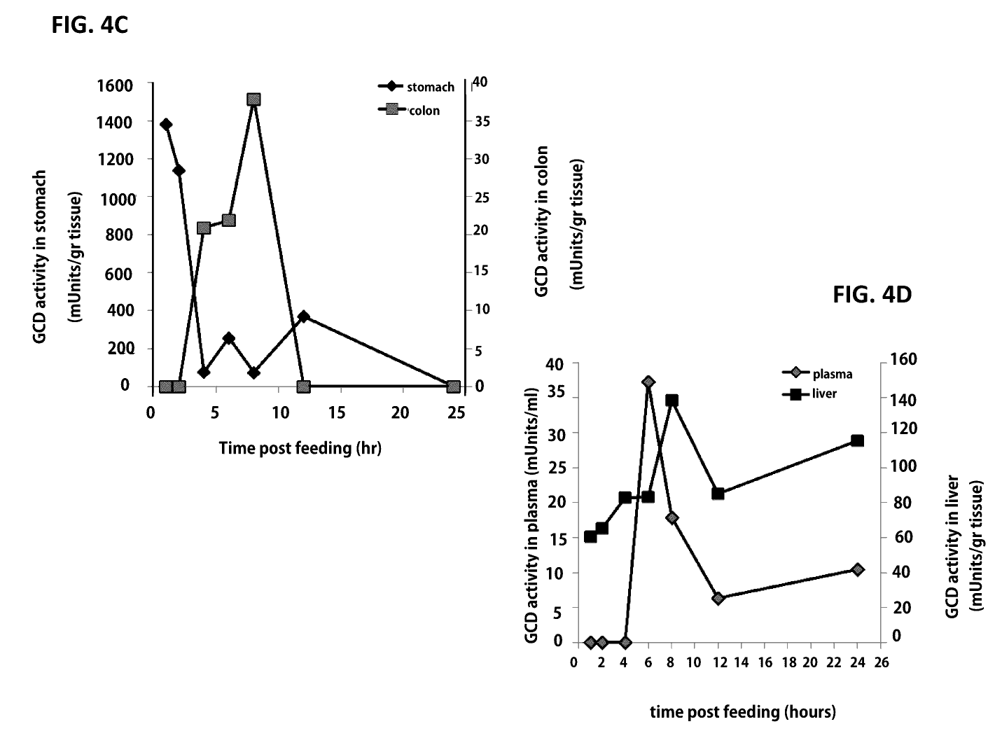

FIGS. 4A-B demonstrate the total stomach content in grams following a gavage feeding with carrot cells overexpressing GCD. The rat stomach loses half of its content after 4-6 hours. FIG. 4C shows the correlation between emptying the GIT and prGCD activity in the stomach and in the colon. While the prGCD activity is reduced in the stomach after 4 hours, the same activity is detected in the colon at 4-8 hours. In accordance, FIG. 4D shows the exogenous GCD activity in the plasma and in the liver following feeding with GCD expressing cells. The peak of GCD activity is reached at 6 and 8 hours post feeding in the plasma and liver, respectively. FIGS. 4C-D, demonstrate the correlation between moving of carrot cells through the GIT and GCD activity in the body. GCD is active along the GI tract and in the target organs as assayed in the liver. The figures demonstrate the slow release characteristics of the carrot cells for the first time enabling oral administration of lower dosage than the extrapolated dosage figured from the bolus IV injection.

### Example 5

### prGCD Activity is Maintained in Carrot Cells Under a Wider pH Range as Compared to the Naked Enzyme

Based on above observations, the present inventors assayed the resistance of prGCD to the extreme environment of the gastric fluid. Purified prGCD and prGCD in carrot cells were treated with:
1. Simulating gastric fluid (including: sodium chloride 70 mM, potassium chloride 50 mM, D-glucose 2.2 mM, pepsin 0.14 mM, Lactic acid 1.1 mM, thiocyanate 1.5 mM and catechin 0.14 mM)
2. pH gradient (1.2-6.0)
3. Extensive shaking at 37° C. for 1, 10, 30 minutes

The cells were then extracted and their prGCD presence was evaluated using western blot analysis with anti prGCD antibodies raised in rabbits (previously described).

Results

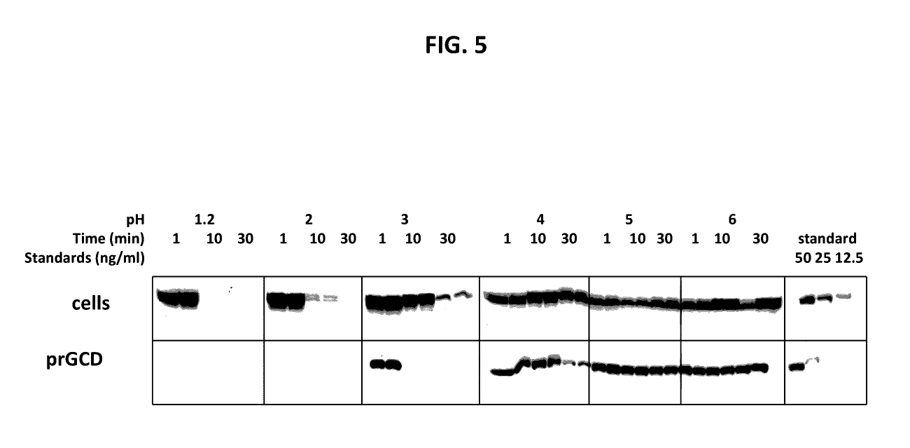

FIG. 5 shows the superiority of plant cells in conferring resistance. Clearly carrot cells over expressing prGCD can be administered on an empty stomach but administration over a light meal can be advantageous.

### Example 6

### prGCD is Released From the Cells Upon Exposure to Simulated Intestinal Fluid Media Containing Pancreatic Enzymes

Carrot cells expressing prGCD were treated with Simulated gastric fluid pH 4 (described above), 10 minutes, shaking at 37° C. and then the medium was removed and the cells were treated with simulated intestinal fluid media, after a fast or after a meal (the exact contents are depicted in table 4 below). The cells were intensively shaken for 30, 60 or 120 minutes. The cells were then separated from the medium and both medium and cells were tested for GCD activity.

The Simulating intestinal fluid included the contents listed in the Table 4, below:

TABLE 4

| Simulating intestinal fluid: | Fasted | Fed |
|---|---|---|
| Monobasic potassium phosphate | 0.049M | 0.049M |
| Sodium hydroxide | 0.0154M | 0.0154M |
| KCl | 0.2M | 0.2M |
| Sodium taurocholate | 3 mM | 15 mM |
| Lecithin | 1.5 mM | 1.5 mM |
| Pancreatin (enzymes) | 70 mg | 1 gr |
| pH | 6.0 | 6.8 |

Results

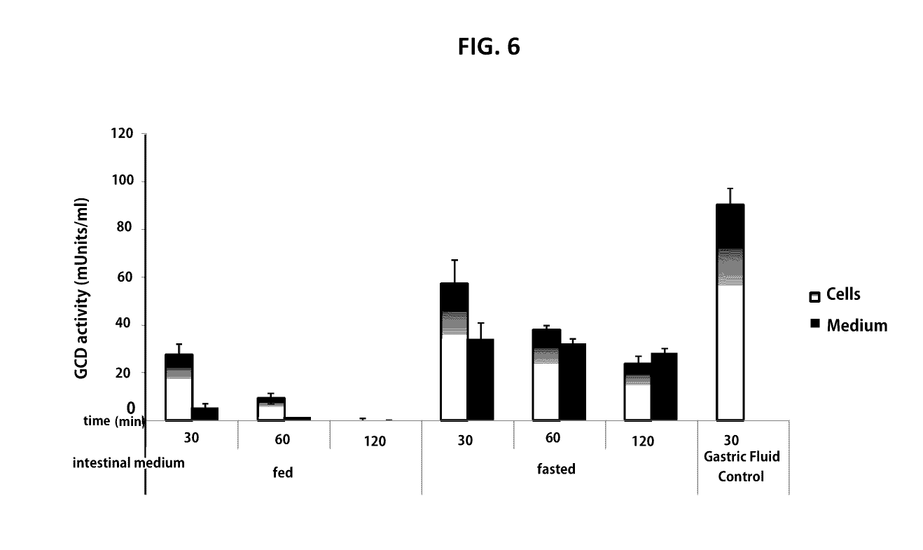

FIG. 6 shows that GCD is released to the medium after exposure to both fed and fasted intestinal fluids but is protected from degradation in the Pancreatin-poor medium corresponding to a light meal environments.

### Example 7

### prGCD Reaches Target Organs Following Feeding in Rats

The experimental procedure is listed in Table 5 below. Feeding dose is an average of the total amount of consumed GCD units as measured for each rat individually.

TABLE 5

| Group | N | Compound | GCD dose | Administration | Time of terminatior |
|---|---|---|---|---|---|
| 1 | 6 | Lyophilized | 0 | 2 feedings | 2 h after |
| | | Carrot (−) cells | | within 6 hours | second feeding |
| 2 | 6 | Lyophilized | 190 Units/Kg body | 2 feedings | 2 h after |
| | | Carrot (prGCD) cells | weight | within 6 hours | second feeding |
| 3 | 6 | prGCD | 170 Units/Kg body | Injection | 1 hour |
| | | | weight | | post injection |

Results

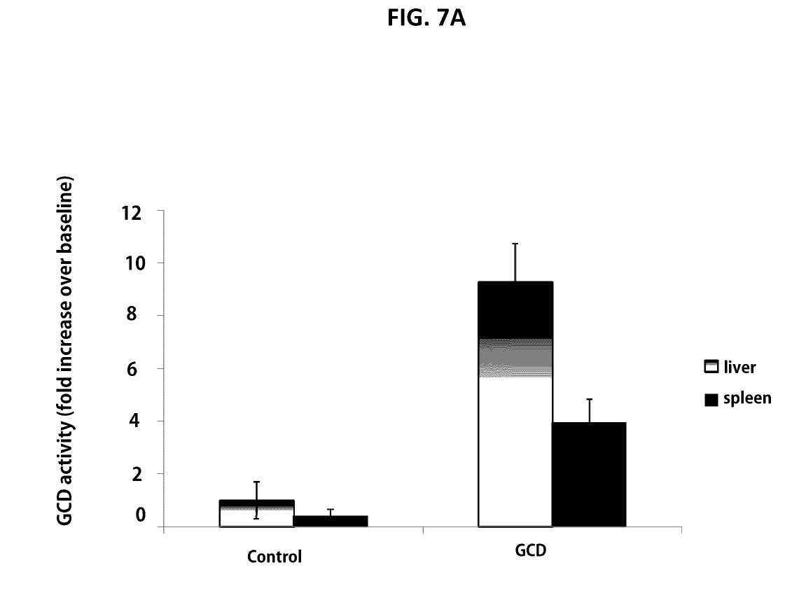

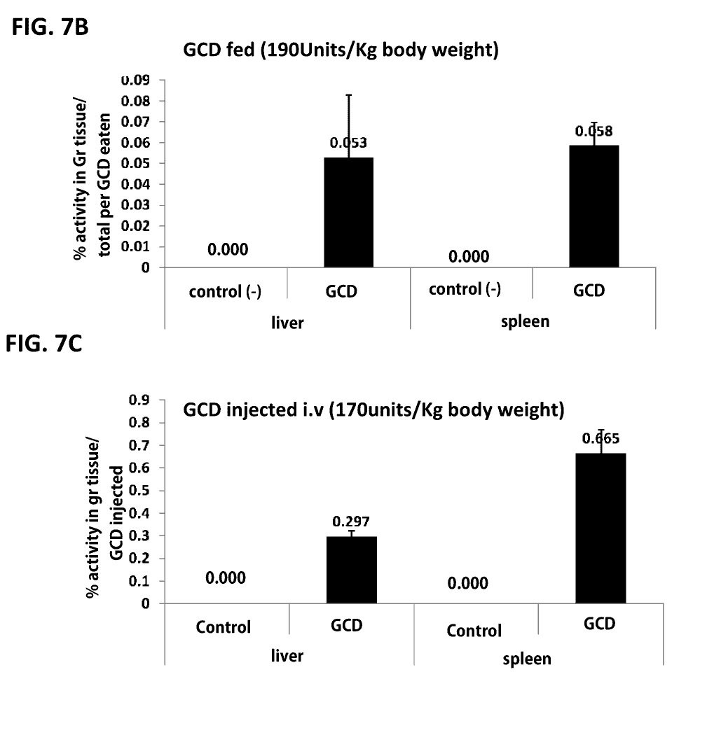

FIG. 7A shows that active prGCD can be detected in the target organs, e.g spleen and liver 2 hours following feeding.

In order to compare between orally administered and IV injected prGCD the percentage of active prGCD that reached target organs, out of the total GCD consumed or administered was measured 2 hours after feeding or 1 hour after injection. The results are shown in FIGS. 7B-C and in Table 6, below. The results are normalized to the amount of active prGCD eaten (FIG. 7B) or injected (FIG. 7C).

TABLE 6

| Spleen | Injection | Feeding |
|---|---|---|
| Given | 170 Units/kg | 190 Units/kg |
| | body weight | body weight |
| Measured in spleen | 0.6% | 0.06% |
| Measured in liver | 0.3% | 0.05% |

These results indicate that 10 times more GCD is required in feeding than that required via injection.

### Example 8

### Pharmacokinetics of Orally Administered prGCD in Rodents

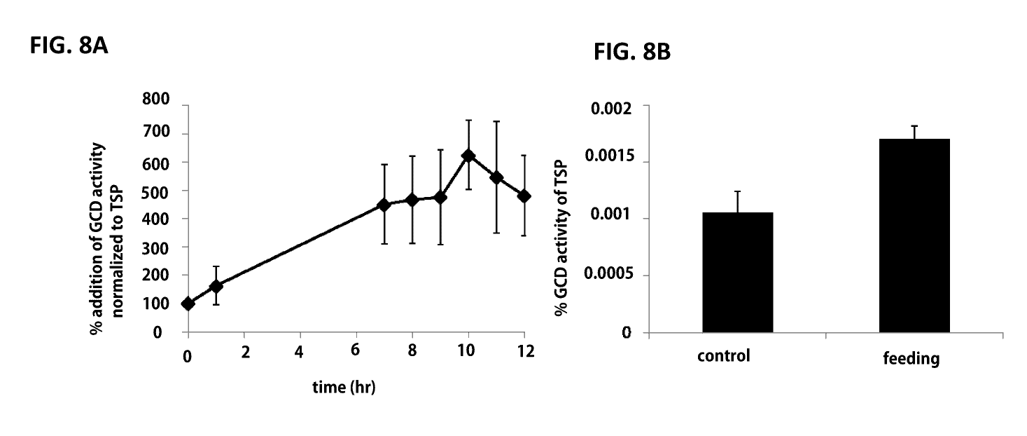

Rats (n=21) were fed with carrot cells twice with a six hours interval. Whole blood (200 ul) was sampled at various time points as indicated from time 0 to 12 hours post feeding. Three samples from different rats at the same time point were pooled. Red blood cells were lysed with 1.2 ml of salt buffer solution (150mM NH4Cl, 10 mM NH4HCO3, 0.1 mM EDTA) for 10 minutes on ice. The leukocytes were washed twice with the salt buffer solution before extraction with 150 μl of GCD activity buffer (0.125% sodium taurocholate, 60 mM phosphate citrate, 0.15% Triton-X100), 10 minutes in TissueLyzer II (Qiagen) with 1 large bead followed by a 10 minute centrifugation at 13,500 rpm. The leukocytes extracts were tested for GCD activity by the 4-Methylumbelliferyl β-D-glucopyranoside (4-MU, Sigma, M3633) assay (ref: Urban D J et al, Comb Chem High Throughput Screen. 2008 December; 11(10):817-24) and normalized to total soluble proteins that were tested using the Bradford assay (FIG. 8, panel A). The rats were then sacrificed and their livers were extracted and analyzed for GCD activity, compared with naïve rats (n=3, FIG. 8B).

### Example 9

### Pharmacokinetics of Orally Administered prGCD in Swine

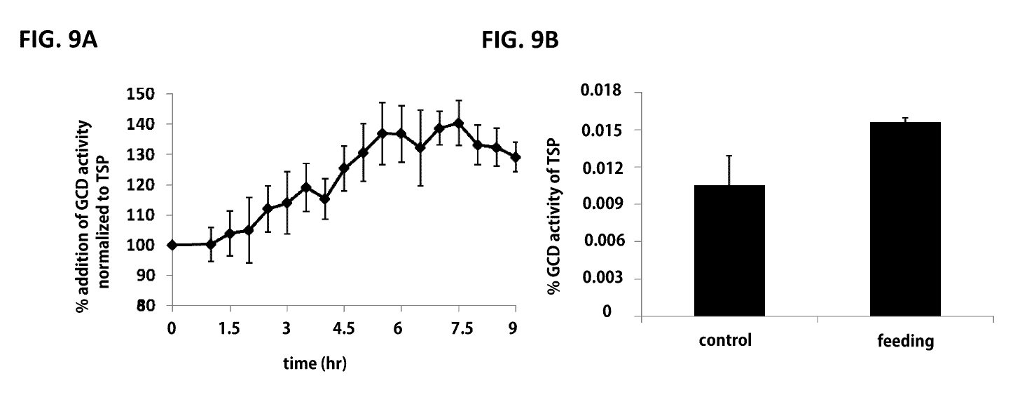

Pigs (n=3) were fed once with carrot cells. Plasma samples (2 ml) were collected at various time points from time 0 to 9 hours post feeding, as indicated. The plasma was then analyzed for GCD activity by the 4-Methylumbelliferyl β-D-glucopyranoside (4-MU, Sigma, M3633) assay (ref: Urban D J et al, Comb Chem High Throughput Screen. 2008 December; 11(10):817-24) and normalized to Total soluble proteins that were tested using the Bradford assay (FIG. 9A). The pigs were then sacrificed and their livers were extracted and tested for GCD activity, compared with naïve pigs (n=5, FIG. 9B).

### Example 10

### Calculation of the Required Dose of GCD in Cells

Oral dosage (U) is calculated from the IV dosage (Zᵢᵥ) adjusted to the prGCD expression rate in carrot cells (X) and adjusted to the measured Bioavailability (F). The oral dosage is given in gram cells per kilogram body weight.

1. Obtaining AUC (Prophetic) of I.V Administration

Rats or pigs are IV injected with 1, 2.5, 10, 15, 30, 60, 100 and 120 units/kg body weight in their tail vein. Whole blood (200 ul) is sampled at various time points e.g. 1, 2, 5, 10, 30, 60, 90, 120 and 240 minutes post injection. Three samples from different rats at the same timepoint are pooled. Red blood cells are lysed with 1.2 ml of salt buffer solution (150mM NH₄Cl, 10 mM NH₄HCO₃, 0.1 mM EDTA) for 10 minutes on ice. The leukocytes are washed twice with the salt buffer solution before extraction with 150 ul of GCD activity buffer (0.125% sodium taurocholate, 60 Mm phosphate citrate, 0.15% Triton-X100), 10 minutes in TissueLyzer II (Qiagen) with 1 large bead followed by a 10 minutes centrifugation at 13500 rpm. The leukocytes extractions are then tested for GCD activity by the 4-Methylumbelliferyl β-D-glucopyranoside (4-MU, Sigma, M3633) assay (ref: Urban D J et al, Comb Chem High Throughput Screen. 2008 December; 11(10):817-24). Total soluble proteins are assayed to normalize the extraction and tested using the Bradford assay.

2. Obtaining AUC (Prophetic) of Oral Administration

Rats or pigs are fed with 0.2, 0.5, 0.75, 1, 1.5, 2, 3, 4, 5 gr carrot cells expressing GCD/Kg body weight once for one hour. Whole blood (200 ul) is sampled at various time points e.g. 1, 2, 3, 4, 5, 6, 7, 8, 9, 10, 11, 12, 14, 16, 18, 24 hours post injection. Three samples from different animals at the same time point are pooled. Blood is then treated and tested the same as in the IV injection.

3. The data obtained from both IV and oral administration of each dose is plotted as GCD activity versus time and the Area Under the Curve (AUC) is calculated.

4. Bioavailability Calculation

Bioavailability is defined as rate and extent of drug input into the systemic circulation i.e. the fraction or percent of the administered dose absorbs intact (as compared to IV administration). (Reference: Clinical Pharmacokinetics Concepts and Applications. Malcolm Rowland and Thomas N. Tozer third edition Lippincott Williams and Wilkins, 1995]

The bioavailability of orally administered GCD is calculated relative to the absorbance of IV administered GCD:

$$AUC - F = \frac{{AUC}_{iv}}{{AUC}_{oral}} x \frac{{dose}_{oral}}{{dose}_{iv}}$$

area under the curve obtained from the pharmacokinetic studies

5. Calculation of the Required Units for Oral Administration

Zᵢᵥ—Required units administered by IV: (units/Kg body weight/day)

Zₒᵣₐₗ—Required units to be administered orally (units/Kg body weight /day)

F=bioavailability

6. Calculation of the Mass of Cells Needed to Reach the Required GCD Unit Oral Dosage:

$${U}_{( gr cells \text{/} Kg body weight} = \frac{{Z}_{iv ( units \text{/} Kg body weight )}}{{FX}_{( units \text{/} gr cells )}}$$

U=Oral calculated dosage (gr cells/Kg body weight)
If required to be given more than once daily U can be further divided to parts (U/2—for twice daily U/4 for four times daily, etc.
Dose can be adjusted individually by for example giving a higher initial dose followed by long term lower doses
The combination of enzyme absorption through the oral route and administration of small amounts daily (vs bi weekly administration of high concentration) is closer in mechanism of slow release regimen. Thus, this regimen might potentially require less enzyme to achieve the therapeutic effect.
personalized medicine to the patent—each patient can easily adjust the regimen, when delivered orally.

### Example 11

### Administration of GCD in Cells in the Clinical Context

In order to assess safety of oral administration of plant recombinant GCD in cells, and to evaluate pharmacokinetic parameters of the plant recombinant GCD after oral administration of GCD in cells in Gaucher's patients, oral dosage of the GCD in cells was provided to Gaucher's patients in a phase I clinical study.

Type of Study: A Phase I exploratory, open-label study to evaluate the safety and pharmacokinetics of oral prGCD (plant recombinant human glucocerebrosidase) in Gaucher patients.

Patient Population

Inclusion Criteria
1. Males and females, 18 years or older
2. Historical diagnosis of Gaucher disease by low leukocyte GCD activity level
3. No quantitable GCD levels in plasma
4. Platelets count <100,000 mm³ (for thrombocytopenia analyses)
5. Body Mass Index (BMI) 19 to 25 kg/m2 (inclusive).
6. Subjects in generally good health in the opinion of the investigator as determined by medical history, vital signs and a physical examination.
7. Negative hepatitis B or hepatitis C serology tests at screening.
8. Ability to provide a written informed consent
9. Female subjects of child-bearing potential or male subjects with female partners of child-bearing potential must agree to use two methods of contraception, one of which must be a barrier method. Acceptable methods of contraception include hormonal products, intrauterine device, or male or female condoms.

Exclusion Criteria
1. Presence of any metabolic or cardiovascular co-morbidity
2. Quantitable levels of GCD in plasma
3. Presence of any GIT disease or symptomatology suspected to be GIT related using a study specific GI questionnaire
4. Subjects with any history of allergic response to drugs or other allergies deemed clinically significant or exclusionary for the study, including known food allergies
5. History of alcohol or drug abuse
6. Subjects who donated blood in the three months, or received blood or plasma derivatives in the six months, preceding study drug administration.
7. Use of any investigational drug at screening or within 3 months of dosing.
8. Subjects with an inability to communicate well with the investigators and study staff (i.e., language problem, poor mental development or impaired cerebral function).

9. Subjects that have difficulty fasting or consuming the standard meals that will be provided.
10. Subjects who are non-cooperative or unwilling to sign the consent form.
11. Pregnant or nursing or planning to be pregnant during the study period.
12. Have used any medication (excluding acetaminophen or dypirone), within 7 days of study drug administration including laxatives or other drugs, teas or food additives known to be used to treat constipation or diarrhea.
13. Presence of any medical, emotional, behavioral or psychological condition that in the judgment of the investigator would interfere with the subject's compliance with the requirements of the study.

Dosing of GCD in cells: Study participants received a single dose of 250 ml of resuspended lyophilized carrot cells expressing GCD comprising 250 units, 500 units or 750 units of GCD, administered orally in pineapple flavored liquid. Following a single dosing, parameters of safety and pharmacokinetics were evaluated in blood samples from the subjects. Dosing was then repeated for three consecutive days.

Assessment of Safety: Adverse events following initial administration, either spontaneously reported or identified during physical examination or clinical laboratory testing, were monitored up to five days after initial administration. Monitoring of adverse events was done from the time of first study drug administration until the end of study.

Pharmacokinetics: Leukocyte GCD activity was measured in the samples of the subjects' blood taken at selected time points during the period from beginning of administration to 30 hours afterwards.

GCD Activity Assay—The GCD enzyme catalyses the cleavage of the 4-methylumbelliferyl-β-D-glucopyranoside substrate, which produces a fluorescent product, 4-methylumbelliferone (4MU), that can be measured.

In this assay, mononuclear cell lysates isolated from peripheral blood were incubated (37° C.) with 4-methylumbelliferyl-β-D-glucopyranoside. Following reaction termination with NaOH, the fluorescent response was measured with excitation of 370 nm and emission of 440 nm to obtain Relative Fluorescent Units (RFU). Each sample concentration (ng/mL) was calculated using a calibration curve prepared with prGCD.

Monitoring of pharmacokinetics was done following initial dose, or following the latter dosings. Pharmacokinetic parameters assessed include the area under the curve “AUC” (see Example 10) for GCD level in the mononuclear cell lysates (AUCO-30 hours) and the maximum concentration (Cmax) of GCD in the mononuclear cell lysates samples from administration to 30 hours afterwards.

Results

Safety Analysis

Tables 7A-B, below, show that the drug was well tolerated. No drug related serious adverse reactions have been reported. Accordingly, no patient discontinued the study prematurely. The treatment didn't induce antibodies.

Serum samples were collected from subjects enrolled in the current study, before and after oral administration of prGCD and were analyzed for the presence of IgG anti-drug antibodies (ADA) using a validated enzyme-linked immunosorbent assay (ELISA) in accordance with common industry procedures.

TABLE 7A

| | No. of AEs | | |
|---|---|---|---|
| | (No. of | % of | |
| Relatedness | Patients) | TotalAEs | AE (#patients) |
| Not related to | 7 (5) | 63.6% | |
| treatment | | | |
| Possibly related to | 3 (2) | 27.3% | Mild dizziness (1) |
| treatment | | | Dizziness (1) |
| Related to treatment | 1 (1) | 9.1% | Nausea (1) |

TABLE 7B

| | # of AEs | AEs |
|---|---|---|
| Severity | (# of patients) | % of Total |
| Adverse events | 11 (7) | |
| Mild or moderate | 11 (7) | 100.0% |
| Severe or very severe | 0 (0) | 0.0% |

AE-adverse event
One SAE (severe adverse event) was non-treatment related (genital herpes-hospitalization);
All related AEs were mild and moderate and recovered without sequelae

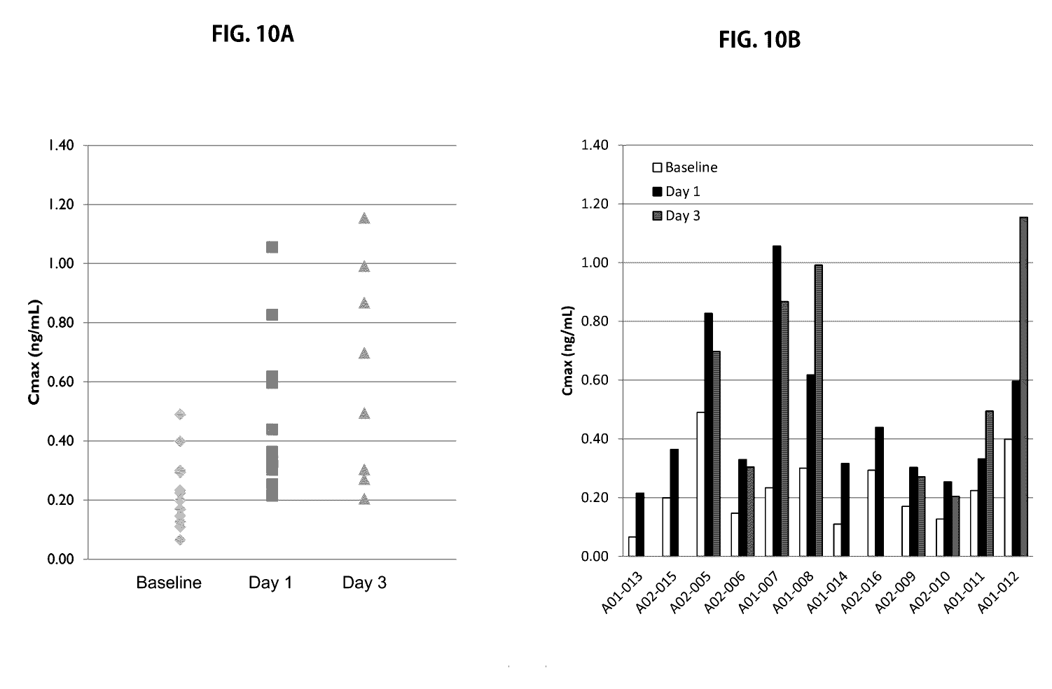

FIGS. 10A-B show the GCD activity in leukocytes following orally delivered prGCD (either 250 U, 500 U or 750 U). As can be seen, GCD activity increased from baseline to day 1 or day 3.

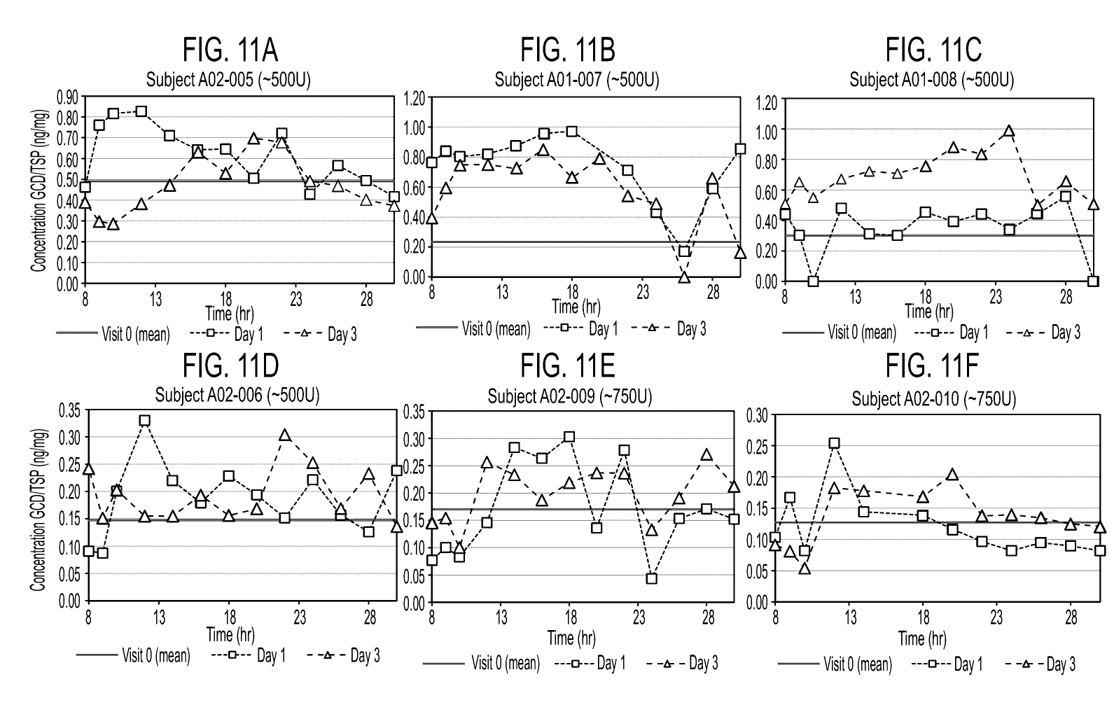

Pharmacokinetics was determined over 30 hours following day 1 and following day 3. The results are shown for representative patients administered with either 500 U or 750 U in FIGS. 11A-F. As can be seen, the active enzyme was detected in patient's blood circulation following oral administration. The observed increase in Cmax of active GCD in leukocytes constitutes evidence of absorption of prGCD after oral administration. Interestingly, the PK profile is different than I.V. administered enzyme (in enzyme replacement therapy, ERT), continuous secretion over about 30 hours versus minutes, it can be hypothesized that the enzyme is protected from degradation by the acidic environment of the GI tract by the encapsulating carrot cells, and thus is secreted into the circulation in a slow release like manner. Thus, C max analysis showed an average increase of over 100% in enzymatic activity from base line, with an increase ranging from approximately 50% to 350% among the different, individual patients in the study. In general, the PK profile of Oral GCD has a pattern of continuous enzyme presence over approximately 30 hours from administration.

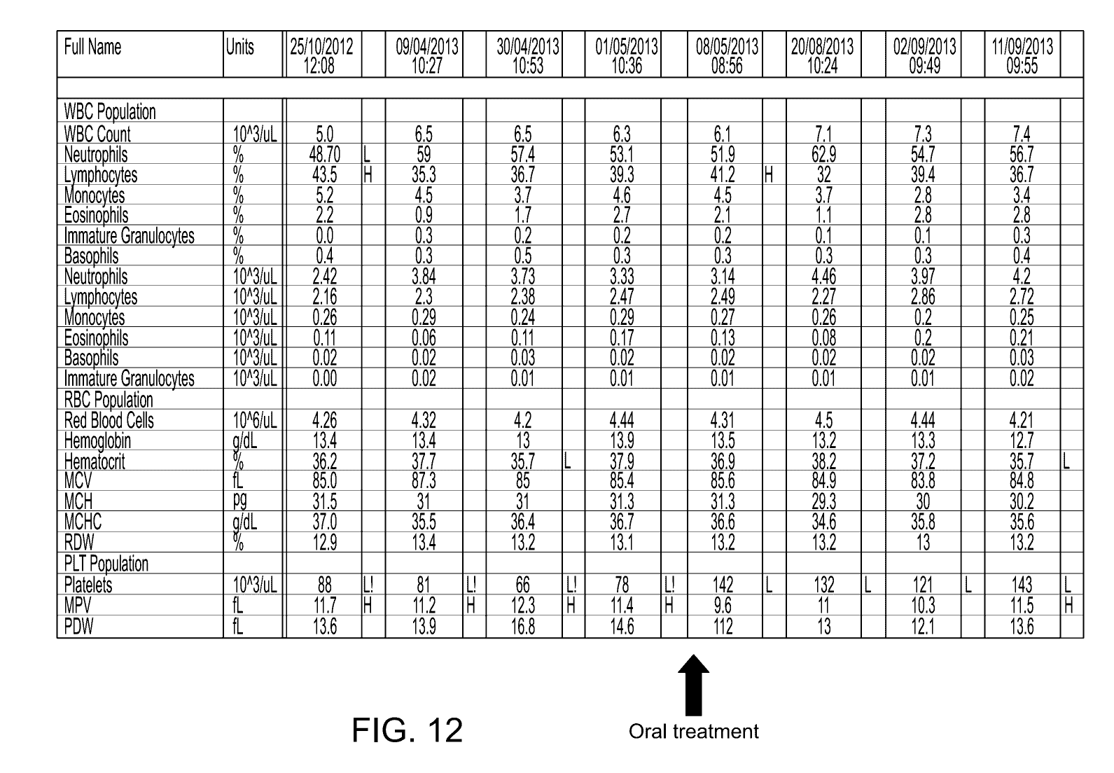

FIG. 12 shows complete blood count of a Gaucher patient performed during more than 6 months prior to treatment initiation according to the teachings of the present invention with oral GCD (250 U) and 4 months thereafter. As can be seen, the patient's platelet exhibited a clinically significant improvement in platelet count, and additionally in white blood cell count,

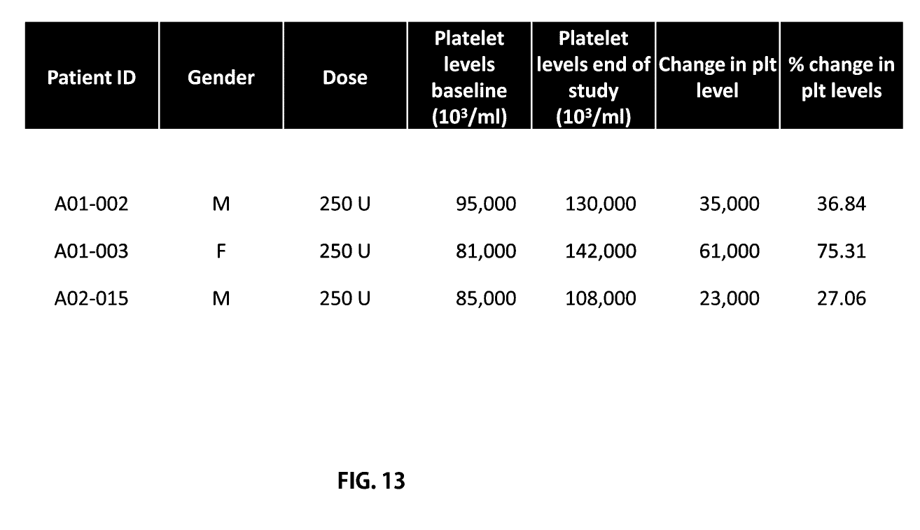

Note, platelet levels in selected thrombocytopenic patients showed meaningful increase after treatment with oral GCD (250 U or 500 U, FIG. 13).

Noteworthy is patient A02-015, a female subject receiving low dose oral prGCD (250 U). This patient has been treated with ERT. Baseline platelet counts were below normal i.e., 85,000/μL. Following oral treatment with prGCD her platelet levels increased by 27% to 108,000/μL.

Conclusions:

Presence of an active enzyme was detected in patient's blood circulation following oral administration. Oral GCD was found to be safe and well tolerated in all 16 patients across all three doses tested, i.e., 250 U, 500 U and 750 U.

Thrombocytopenic patients exhibited meaningful improvement in platelet count.

Importantly, ERT-non responsive patients, i.e., those patients which do not adequately respond to ERT in terms of platelet counts can benefit from the present treatment which seems efficacious in restoring the platelet counts even after short term treatment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of maintaining disease stability in a subject having Gaucher's disease following switch from enzyme replacement therapy (ERT), the method comprising orally administering to the subject a therapeutically effective amount of recombinant glucocerebrosidase (GCD) comprised in plant cells, thereby maintaining disease stability following switch.

2. (canceled)

3. The method of claim 1, further comprising periodically testing at least one of liver volume, spleen volume and complete blood count.

4. The method of claim 1, wherein the subject has been treated with ERT for at least 2 years, with a stable dose regimen for at least last 6 months before said switch.

5. The method of claim 1, wherein the subject exhibits clinically and biologically stable disease for the last 12 months prior to said switch as manifested by stable organomegaly, no progressive symptomatic documented bone disease, no major surgery, hemoglobin level >11 g/dl, mean platelet count >120,000/μL±40%, mean platelet count ≦120,000/μL±20%, chitotriosidase activity within 20% of the mean, no blood transfusion or major bleeding, no acute avascular necrosis event.

6. The method of claim 5, wherein said stable organomegaly is manifested by liver volume within 10% of the mean and spleen volume within 10% of the mean.

7. The method of claim 1, wherein the Gaucher's disease is type I or type III Gaucher's disease.

8. The method of claim 1, wherein said therapeutically effective amount of GCD corresponds to 1-1920 units/Kg/14 days.

9. The method of claim 1, wherein said therapeutically effective amount of GCD corresponds to 50-150 units/Kg/day, thereby treating Gaucher's disease.

10. The method of claim 1, wherein said administering is performed preprandially or over a light meal such that the stomach pH is above 2, thereby treating Gaucher's disease.

11. The method of claim 1, wherein said administering is effected daily.

12. The method of claim 1, wherein said administering is performed preprandially.

13. The method of claim 1, wherein said administering is effected following light meal such that the stomach pH of said subject is above 2.

14. The method of claim 1, wherein said cells comprise carrot cells.

15. The method of claim 1, wherein said administering is performed once a day, twice a day, three times a day or four times a day.

16-18. (canceled)

19. The method of claim 1, wherein said plant cells comprise lyophilized plant cells.

20. The method of claim 1, wherein said glucocerebrosidase is human glucocerebrosidase.

21. (canceled)

22. The method of claim 1, wherein said human glucocerebrosidase protein is linked at its N terminus to an endoplasmic reticulum signal peptide.

23. (canceled)

24. The method of claim 1, wherein said human glucocerebrosidase protein is linked at its C terminus to a vacuolar signal peptide.

25. (canceled)

26. The method of claim 1, wherein said glucocerebrosidase has an increased affinity for, and uptake into macrophages, in comparison with the corresponding affinity and uptake of a recombinant human glucocerebrosidase protein produced in mammalian cells, and having glucocerebrosidase catalytic activity.

27. The method of claim 1, wherein the main glycan structure of said glucocerebrosidase of said plant cells comprises at least one xylose residue and at least one exposed mannose residue, as measured by linkage analysis.
